SEQUENCE LISTING (48 sequences)

SEQ ID NO: 1 - protein, 8 aa, Mus musculus
GFTFSDAW

SEQ ID NO: 2 - protein, 10 aa, Mus musculus
IRDETNNHIT

SEQ ID NO: 3 - protein, 6 aa, Mus musculus
AAGFAY

SEQ ID NO: 4 - protein, 11 aa, Mus musculus
QSLEKSNGKTY

SEQ ID NO: 5 - skipped

SEQ ID NO: 6 - protein, 9 aa, Mus musculus
LQVSHVPFT

SEQ ID NO: 7 - protein, 9 aa, Mus musculus
GYSITSGYS

SEQ ID NO: 8 - protein, 7 aa, Mus musculus
IHYSGRT

SEQ ID NO: 9 - protein, 6 aa, Mus musculus
ARSMDY

SEQ ID NO: 10 - protein, 6 aa, Mus musculus
QDIKTY

SEQ ID NO: 11 - skipped

SEQ ID NO: 12 - protein, 9 aa, Mus musculus
LQHGESPPT

SEQ ID NO: 13 - protein, 8 aa, Mus musculus
GFTFSDAW

SEQ ID NO: 14 - protein, 10 aa, Mus musculus
IRSKAKNHIT

SEQ ID NO: 15 - protein, 6 aa, Mus musculus
TGGFAY

SEQ ID NO: 16 - protein, 11 aa, Mus musculus
QSLEKSNGNTY

SEQ ID NO: 17 - skipped

SEQ ID NO: 18 - protein, 9 aa, Mus musculus
VQVSHVPFT

SEQ ID NO: 19 - protein, 115 aa, Mus musculus
AVNLEESGGGLVLPGGSMKLSCTASGFTFSDAWMDWVRRSPEKGLEWLAEIRDETNNHITYYAESVKVRFIISRDDSKSSVYLQMNNLRPEDTGIYYCAAGFAYWGQGTLVTVSA

SEQ ID NO: 20 - protein, 112 aa, Mus musculus
DAVMTQTPLSLSVSLGDQASISCRSSQSLEKSNGKTYLNWYLQKPGQSPQLLIYRVSNRFSGVLDRFSGSGSGTDFTLKISRVEAEDLGLYFCLQVSHVPFTFGSGTKLEIK

SEQ ID NO: 21 - protein, 113 aa, Mus musculus
DVQLQESGPDLVKPSQSLSLTCTVTGYSITSGYSWHWIRQFPGNKLEWMGYIHYSGRTNYNPSLKSRISITRDTSKNQFFLQLNSVTTEDTATYYCARSMDYWGQGTSVSVSS

SEQ ID NO: 22 - protein, 107 aa, Mus musculus
DIKMTQSPSSMYASLGERVTITCKASQDIKTYLNWYQQKPWKSPKTLIYYATSLADGVPSRFSGSGSGQDFSLTISSLESDDTATYFCLQHGESPPTFGGGTKLEIK

SEQ ID NO: 23 - protein, 115 aa, Mus musculus
EVKLEESGGGLVQPGGSMKLSCAASGFTFSDAWMDWVRRSPEKGLEWIAEIRSKAKNHITYYAESVKGRFTISRDDSKSSVYLQMNSLRTEDSGIYYCTGGFAYWGQGTLVTVSA

SEQ ID NO: 24 - protein, 112 aa, Mus musculus
DAVMTQNPLSLPVSLGDQASISCRSSQSLEKSNGNTYLNWYLQKPGQSPQLLIYRVSNRFSGVPDRISGGGSGTDFTLKISRVEAEDLGVYFCVQVSHVPFTFGSGTKLEIK

SEQ ID NO: 25 - DNA, 24 bp, Mus musculus
  source 1..24: mol_type = other DNA
ggattcacttttagtgacgcctgg

SEQ ID NO: 26 - DNA, 30 bp, Mus musculus
  source 1..30: mol_type = other DNA
attagagacgaaactaataatcatataaca

SEQ ID NO: 27 - DNA, 18 bp, Mus musculus
  source 1..18: mol_type = other DNA
gcggctggatttgcttac

SEQ ID NO: 28 - DNA, 33 bp, Mus musculus
  source 1..33: mol_type = other DNA
cagagccttgaaaagagtaatggaaaaacctat

SEQ ID NO: 29 - skipped

SEQ ID NO: 30 - DNA, 27 bp, Mus musculus
  source 1..27: mol_type = other DNA
ctccaagtttcacatgtcccattcacg

SEQ ID NO: 31 - DNA, 27 bp, Mus musculus
  source 1..27: mol_type = other DNA
ggctactccatcaccagtggttatagc

SEQ ID NO: 32 - DNA, 21 bp, Mus musculus
  source 1..21: mol_type = other DNA
atacactacagtggtcgcact

SEQ ID NO: 33 - DNA, 18 bp, Mus musculus
  source 1..18: mol_type = other DNA
gcaagatctatggactac

SEQ ID NO: 34 - DNA, 18 bp, Mus musculus
  source 1..18: mol_type = other DNA
caggacattaaaacctat

SEQ ID NO: 35 - skipped

SEQ ID NO: 36 - DNA, 27 bp, Mus musculus
  source 1..27: mol_type = other DNA
ctacagcatggtgagagccctcccacg

SEQ ID NO: 37 - DNA, 24 bp, Mus musculus
  source 1..24: mol_type = other DNA
ggattcacttttagtgacgcctgg

SEQ ID NO: 38 - DNA, 30 bp, Mus musculus
  source 1..30: mol_type = other DNA
attagaagcaaagctaaaaatcacataaca

SEQ ID NO: 39 - DNA, 18 bp, Mus musculus
  source 1..18: mol_type = other DNA
acaggggggtttgcttac

SEQ ID NO: 40 - DNA, 33 bp, Mus musculus
  source 1..33: mol_type = other DNA
cagagtcttgaaaaaagtaatggaaacacctat

SEQ ID NO: 41 - skipped

SEQ ID NO: 42 - DNA, 27 bp, Mus musculus
  source 1..27: mol_type = other DNA
gtccaagtttcacatgtcccattcacg

SEQ ID NO: 43 - DNA, 345 bp, Mus musculus
  source 1..345: mol_type = other DNA
gcagtgaatcttgaggagtctggaggaggcttggtgctacctggaggatccatgaaactctcttgtactgcctctggattcacttttagtgacgcctggatggactgggtccgccggtctccagagaaggggcttgagtggcttgctgaaattagagacgaaactaataatcatataacatattatgctgagtctgtgaaagtgaggttcatcatctcaagagatgattccaaaagtagtgtctaccttcaaatgaacaacttaagacctgaagacactggcatttattactgtgcggctggatttgcttactggggccaagggactctggtcactgtctctgca

SEQ ID NO: 44 - DNA, 336 bp, Mus musculus
  source 1..336: mol_type = other DNA
gatgctgtgatgacccaaactccactctccctgtctgtcagtcttggagatcaagcctccatctcttgcaggtctagtcagagccttgaaaagagtaatggaaaaacctatttgaactggtacctccagaaaccaggccagtctccacagctcctgatctatagggtttccaaccgattttctggggtcctagacaggttcagtggtagtggatcagggacagatttcacactgaaaatcagtagagtggaggctgaggatttgggactttatttctgcctccaagtttcacatgtcccattcacgttcggctcggggacaaagttggaaataaaa

SEQ ID NO: 45 - DNA, 339 bp, Mus musculus
  source 1..339: mol_type = other DNA
gatgtgcagcttcaggagtcaggacctgacctggtgaaaccttctcagtcactttcactcacctgcactgtcactggctactccatcaccagtggttatagctggcactggatccggcagtttccaggaaacaaactggaatggatgggctatatacactacagtggtcgcactaactacaacccatctctcaaaagtcgaatctctatcactcgagacacatccaagaaccagttcttcctgcagttgaattctgtgactactgaggacacagccacatattactgtgcaagatctatggactactggggtcaaggaacctcagtctccgtctcctca

SEQ ID NO: 46 - DNA, 321 bp, Mus musculus
  source 1..321: mol_type = other DNA
gacatcaagatgacccagtctccatcctccatgtatgcatcgctgggagagagagtcactatcacttgcaaggcgagtcaggacattaaaacctatttaaactggtaccagcagaaaccatggaaatctcctaagaccctgatctattatgcaacaagcttggcagatggggtcccatcacgattcagtggcagtggatctggacaagatttttctctaaccatcagcagcctggagtctgacgatacagcaacttatttctgtctacagcatggtgagagccctcccacgttcggaggggggaccaaactggaaataaaa

SEQ ID NO: 47 - DNA, 345 bp, Mus musculus
  source 1..345: mol_type = other DNA
gaagtgaagcttgaggagtctggaggaggcttggtgcaacctggaggatccatgaaactctcttgtgctgcctctggattcacttttagtgacgcctggatggactgggtccgccggtctccagagaaggggcttgagtggattgctgaaattagaagcaaagctaaaaatcacataacatactatgctgagtctgtgaaggggaggttcaccatctcaagagatgattccaaaagtagtgtctacctacaaatgaacagcttaagaactgaagactctggcatttattactgtacaggggggtttgcttactggggccaagggactctggtcactgtctctgca

SEQ ID NO: 48 - DNA, 336 bp, Mus musculus
  source 1..336: mol_type = other DNA
gatgctgtgatgacccaaaatccactctccctgcctgtcagtcttggagatcaagcctccatctcttgcaggtctagtcagagtcttgaaaaaagtaatggaaacacctatttgaactggtacctccagaaaccaggccagtctccacagctcctgatatacagggtttccaaccgattttctggggtcccagacaggatcagtggtggtggatcagggacagatttcacactgaaaatcagcagagtggaggctgaggatttgggagtttatttctgcgtccaagtttcacatgtcccattcacgttcggctcggggacaaagttggaaataaaa

(19) United States
(12) Patent Application Publication
(10) Pub. No.: US 2024/0377414 A1
(43) Pub. Date: Nov. 14, 2024

(54) ASSAY METHOD FOR RELAXIN

(71) Applicant: BELLYLABS OY, Helsinki (FI)

(72) Inventors: Anni ESKELI, Helsinki (FI); Mika SARAMÄKI, Karkkila (FI)

(21) Appl. No.: 18/691,851

(22) PCT Filed: Sep. 13, 2022
(86) PCT No.: PCT/FI2022/050616

§ 371 Date: Mar. 13, 2024

(30) Foreign Application Priority Data
Sep. 14, 2021 (FI) 20215970

(51) Int. Cl. G01N 33/68; C07K 16/26

(52) U.S. Cl. CPC G01N 33/689; C07K 16/26; C07K 2317/92; G01N 2333/64

(57) ABSTRACT

The present invention relates to pregnancy testing in animals such as dogs and cats. In particular, the present invention provides assay methods for relaxin, and a kit for pregnancy testing in an animal, anti-relaxin antibodies and functional fragments thereof which can be used in the assay, hybridoma cell lines producing the same, nucleic acids encoding the same, and methods for producing the anti-relaxin antibodies and functional fragments thereof.

### FIELD OF THE INVENTION

The present invention relates to pregnancy testing in animals such as dogs and cats. In particular, the present invention provides assay methods for relaxin, and a kit for pregnancy testing in an animal, anti-relaxin antibodies and functional fragments thereof which can be used in the assay, hybridoma cell lines producing the same, nucleic acids encoding the same, and methods for producing the anti-relaxin antibodies and functional fragments thereof.

### BACKGROUND OF THE INVENTION

Pregnancy testing in animals such as cats and dogs is often performed to differentiate between a successful breeding and a pseudopregnancy as well as to for example adapt the diet of the pregnant female to meet the needs of the developing offspring. Pregnancy in a female dog or cat begins following a successful breeding or artificial insemination. The length of gestation in a dog is on average 63 days and between 60 to 65 days in a cat. Occasionally, the female dog or cat may show symptoms of pseudopregnancy including weight gain, mild lethargy, milk production and nesting behaviour, even if they are not mated.

Typically, pregnancy in a dog or a cat is confirmed by a skilled professional such as a veterinarian. During days 26-35 of canine gestation or days 20-30 of feline gestation a veterinarian may confirm pregnancy by palpating the uterus through the abdomen. During this time the gestational sacs are small, approximately 2 centimeters in diameter in dogs, and may be difficult to detect. An incautious or rough palpation may damage the gestational sacs and lead to miscarriage. In both cats and dogs, enlargement and pink colour of the teats and mammary glands may develop during gestation as an external sign of pregnancy but may also be a symptom of pseudopregnancy.

A more accurate, faster and safer method for confirming pregnancy in both dogs and cats is ultrasound which may detect gestational sacs as early as during days 18-20 of gestation. If no puppies or kittens are seen in this early ultrasound, it is typically repeated after 1 week. Typically, the ultrasound is performed on day 28 of gestation in dogs when the specificity of detecting pregnancy is about 99.3%. Performing ultrasound may require that hair is shaved from the animal's abdomen area, causing damage to fur.

Pregnancy testing in animals such as dogs and cats involves several disadvantages. Confirming pregnancy by palpation requires a skilled professional such as a veterinarian, and typically cannot be performed by the animal owner him/herself. In addition, palpation involves a risk of harming the foetuses. Ultrasound requires expensive instrumentation and a skilled professional such as a veterinarian to perform the study. Because of involving a veterinarian, pregnancy testing of animals in general is costly.

Previously, pregnancy tests for animals such as dogs and cats based on detecting relaxin with antibodies have been developed. For example, the ReproCHEK test kit (Synbiotics Corp., USA) is a microwell immunoassay utilizing polyclonal antibodies, which are known to have batch-to-batch performance variability. In addition, the ReproCHEK test involves use of pipets, several reagents and numerous protocol steps, being both laborious and error-prone to perform due to the several stages involved and requirement of technical skill. As a further example, FASTest RELAXIN (Diagnostik Megacor, Hörbranz, Austria) is a pregnancy test for dogs and cats based on monoclonal antibodies. FASTest RELAXIN is suitable for serum and plasma samples but not for whole blood.

There is thus a longstanding need for pregnancy tests for dogs and cats that are affordable, possible to perform even at home and do not necessarily require involvement of a skilled professional.

### BRIEF DESCRIPTION OF THE INVENTION

An object of the present invention is thus to provide a pregnancy test for animals such as cats and dogs. This object is achieved by arrangements which are characterized by what is stated in the independent claims. Some preferred embodiments are disclosed in the dependent claims.

The invention is, at least partly, based on studies revealing that an assay based on certain anti-relaxin antibodies may be used as a pregnancy test for dogs and cats.

More specifically provided is an assay method for relaxin, wherein the method comprises
(a) binding relaxin with a capture antibody comprising
a VL domain comprising a CDR1 region having an amino acid sequence set forth in SEQ ID NO: 10, a CDR2 region having an amino acid sequence set forth in SEQ ID NO: 11, and a CDR3 region having an amino acid sequence set forth in SEQ ID NO: 12, and a VH domain comprising a CDR1 region having an amino acid sequence set forth in SEQ ID NO: 7, a CDR2 region having an amino acid sequence set forth in SEQ ID NO: 8, and a CDR3 region having an amino acid sequence set forth in SEQ ID NO: 9;
or a functional fragment thereof which has independently at least 80% sequence identity, or at least 85%, 90%, 95%, 96%, 97%, 98% or 99% sequence identity to any one of SEQ ID NO: 7-12, wherein the functional fragment is capable of binding relaxin, in particular canine or feline relaxin, preferably canine relaxin;
and
(b) binding relaxin with a first detection antibody comprising
a VL domain comprising a CDR1 region having an amino acid sequence set forth in SEQ ID NO: 4, a CDR2 region having an amino acid sequence set forth in SEQ ID NO: 5, and a CDR3 region having an amino acid sequence set forth in SEQ ID NO: 6, and a VH domain comprising a CDR1 region having an amino acid sequence set forth in SEQ ID NO: 1, a CDR2 region having an amino acid sequence set forth in SEQ ID NO: 2, and a CDR3 region having an amino acid sequence set forth in SEQ ID NO: 3;
or a functional fragment thereof which has independently at least 80% sequence identity, or at least 85%, 90%, 95%, 96%, 97%, 98% or 99% sequence identity to any one of SEQ ID NO: 1-6, wherein the functional fragment is capable of binding relaxin, in particular canine or feline relaxin, preferably canine relaxin;
and
(c) binding relaxin with a second detection antibody comprising
a VL domain comprising a CDR1 region having an amino acid sequence set forth in SEQ ID NO: 16, a CDR2 region having an amino acid sequence set forth in SEQ ID NO: 17, and a CDR3 region having an amino acid sequence set forth in SEQ ID NO: 18, and a VH domain comprising a CDR1 region having an amino acid sequence set forth in SEQ ID NO: 13, a CDR2 region having an amino acid sequence set forth in SEQ ID NO: 14, and a CDR3 region having an amino acid sequence set forth in SEQ ID NO: 15;
or a functional fragment thereof which has independently at least 80% sequence identity, or at least 85%, 90%, 95%, 96%, 97%, 98% or 99% sequence identity to any one of SEQ ID NO: 13-18, wherein the functional fragment is capable of binding relaxin, in particular canine or feline relaxin, preferably canine relaxin.

Also provided is an assay method for relaxin, wherein the method comprises
(a) binding relaxin with a capture antibody comprising
a VH domain having an amino acid sequence set forth in SEQ ID NO: 21, and a VL domain having an amino acid sequence set forth in SEQ ID NO: 22;
or a functional fragment thereof which has independently at least 80% sequence identity, or at least 85%, 90%, 95%, 96%, 97%, 98% or 99% se-quence identity to any one of SEQ ID NO: 21 or 22, wherein the functional fragment is capable of binding relaxin, in particular canine or feline relaxin, preferably canine relaxin;
and
(b) binding relaxin with a first detection antibody comprising
a VH domain having an amino acid sequence set forth in SEQ ID NO: 19, and a VL domain having an amino acid sequence set forth in SEQ ID NO: 20;
or a functional fragment thereof which has independently at least 80% sequence identity, or at least 85%, 90%, 95%, 96%, 97%, 98% or 99% sequence identity to any one of SEQ ID NO: 19 or 20, wherein the functional fragment is capable of binding relaxin, in particular canine or feline relaxin, preferably canine relaxin;
and
(c) binding relaxin with a second detection antibody comprising
a VH domain having an amino acid sequence set forth in SEQ ID NO: 23, and a VL domain having an amino acid sequence set forth in SEQ ID NO: 24;
or a functional fragment thereof which has independently at least 80% sequence identity, or at least 85%, 90%, 95%, 96%, 97%, 98% or 99% sequence identity to any one of SEQ ID NO: 23 or 24, wherein the functional fragment is capable of binding relaxin, in particular canine or feline relaxin, preferably canine relaxin.

In this context further provided is a kit for pregnancy testing in an animal, wherein the kit comprises the capture antibody and the detection antibodies or functional fragments thereof as defined in the preceding methods, optionally wherein the animal is a dog or a cat.

Forming part of such a kit, further provided is an anti-relaxin antibody or functional fragment thereof, wherein said antibody or functional fragment comprises
(i) a VL domain comprising a CDR1 region having an amino acid sequence set forth in SEQ ID NO: 10, a CDR2 region having an amino acid sequence set forth in SEQ ID NO: 11, and a CDR3 region having an amino acid sequence set forth in SEQ ID NO: 12, and a VH domain comprising a CDR1 region having an amino acid sequence set forth in SEQ ID NO: 7, a CDR2 region having an amino acid sequence set forth in SEQ ID NO: 8, and a CDR3 region having an amino acid sequence set forth in SEQ ID NO: 9;
or a functional fragment thereof which has independently at least 80% sequence identity, or at least 85%, 90%, 95%, 96%, 97%, 98% or 99% se-quence identity to any one of SEQ ID NO: 7-12, wherein the functional fragment is capable of binding relaxin, in particular canine or feline relaxin, preferably canine relaxin;
or
(ii) a VL domain comprising a CDR1 region having an amino acid sequence set forth in SEQ ID NO: 4, a CDR2 region having an amino acid sequence set forth in SEQ ID NO: 5, and a CDR3 region having an amino acid sequence set forth in SEQ ID NO: 6, and a VH domain comprising a CDR1 region having an amino acid sequence set forth in SEQ ID NO: 1, a CDR2 region having an amino acid sequence set forth in SEQ ID NO: 2, and a CDR3 region having an amino acid sequence set forth in SEQ ID NO: 3;
or a functional fragment thereof which has independently at least 80% sequence identity, or at least 85%, 90%, 95%, 96%, 97%, 98% or 99% se-quence identity to any one of SEQ ID NO: 1-6, wherein the functional fragment is capable of binding relaxin, in particular canine or feline relaxin, preferably canine relaxin;
or
(iii) a VL domain comprising a CDR1 region having an amino acid sequence set forth in SEQ ID NO: 16, a CDR2 region having an amino acid sequence set forth in SEQ ID NO: 17, and a CDR3 region having an amino acid sequence set forth in SEQ ID NO: 18, and a VH domain comprising a CDR1 region having an amino acid sequence set forth in SEQ ID NO: 13, a CDR2 region having an amino acid sequence set forth in SEQ ID NO: 14, and a CDR3 region having an amino acid sequence set forth in SEQ ID NO: 15;
or a functional fragment thereof which has independently at least 80% sequence identity, or at least 85%, 90%, 95%, 96%, 97%, 98% or 99% se-quence identity to any one of SEQ ID NO: 13-18, wherein the functional fragment is capable of binding relaxin, in particular canine or feline relaxin, preferably canine relaxin.

Further provided is an anti-relaxin antibody or functional fragment thereof, wherein said antibody or functional fragment comprises
(i) a VH domain having an amino acid sequence set forth in SEQ ID NO: 19, and a VL domain having an amino acid sequence set forth in SEQ ID NO: 20, or
(ii) a VH domain having an amino acid sequence set forth in SEQ ID NO: 21, and a VL domain having an amino acid sequence set forth in SEQ ID NO: 22, or
(iii) a VH domain having an amino acid sequence set forth in SEQ ID NO: 23, and a VL domain having an amino acid sequence set forth in SEQ ID NO: 24;
and wherein the functional fragment thereof has independently at least 80% sequence identity, or at least 85%, 90%, 95%, 96%, 97%, 98% or 99% sequence identity to any one of SEQ ID NO: 19, 20, 21, 22, 23, or 24, and wherein the functional fragment thereof is capable of binding relaxin, in particular canine or feline relaxin, preferably canine relaxin.

Further disclosed is a nucleic acid encoding the anti-relaxin antibody or functional fragment thereof. Optionally, the nucleic acid is cDNA. The nucleic acid may be comprised in a vector or a plasmid. The vector or plasmid may be comprised in a cell to produce the anti-relaxin antibody or functional fragment thereof. Further, a method of preparing the antibody or functional fragment thereof is provided.

The anti-relaxin antibody or functional fragment thereof can be advantageously used in an assay for relaxin.

An advantage of the invention is that a sensitive, easy to use test for early detection of pregnancy in animals such as dogs and cats is provided.

### BRIEF DESCRIPTION OF THE DRAWINGS

In the following the invention will be described in greater detail by means of preferred embodiments with reference to the accompanying drawings, in which

FIG. 1 shows the results of dog pregnancy tests performed with anti-relaxin antibody combinations 2H7-10B11, 2H7-2E12 and 2F10-2H7. In FIG. 1, 1) is the 2H7-2E12 pair with 2H7 as capture and 2E12 as detection antibody, 3) is the 2H7-10B11 pair with 2H7 as capture and 10B11 as detection antibody and 2) is the 2F10-2H7 pair with 2F10 as capture and 2H7 as detection antibody. The upper line is a control line showing that the test has been performed successfully. The samples are from three different dogs (1=Bella, 2=Ebba, 3=Noita) and a control sample (4) which is a PBS buffer solution. Samples 1 and 2 are from pregnant dogs and sample 3 is a negative control from a non-pregnant dog;

FIG. 2 shows the results of dog pregnancy tests performed by pairing capture antibody 2H7 with detection antibody 7H1, 4D7, 4C5, 6E9 or 9B8. The first test strip from the left is a reference antibody pair 2H7-10B11/2E12 i.e. capture antibody 2H7 paired with two detection antibodies 10B11 and 2E12. The sample in all tests is pregnant dog serum sample (Bella);

FIG. 3 shows results of dog pregnancy tests performed with capture antibody—detection antibody pairs 2H7-7G7 (A), 2H7-5H11 (B), 2H7-2A4 (C), 21H 7-2F10 (D) 2E12-3A7 (E), 2E12-10B11/2E12 (F), 2E12-7G7 (G), 2E12-5H11 (H) and 2E12-2F10 (I). Control (1) is 1×PBS buffer and positive sample (2) in all tests is pregnant dog serum (Bella);

FIG. 4 shows results of dog pregnancy tests performed using two antibodies as a detection antibody pair with capture antibody 2H7. The antibody combinations were 2H7-2A4/7H1 (A), 2H7-2A4/9B8 (B) and 2H7-7H1/9B8 (C). Control (1) was 1×PBS buffer, negative control (2) was serum from a non-pregnant female dog (Noita) and positive samples were pregnant dog serums Bella (3), Honey (4) and Macy (5);

FIG. 5 shows test results of FASTest RELAXIN dog pregnancy test with 1:1 dilution (above) and 1:16 dilution (below) of sample. The sample was pregnant dog serum (Ebba, 48 d post-breeding);

FIG. 6 shows results of a dog pregnancy test performed using anti-relaxin antibodies 2A4 and 9B8 as a detection antibody pair with capture antibody 2H7. The sample was pregnant dog serum sample (Ebba, 48 d post-breeding) and sample dilutions 1:1, 1:2, 1:4 1:8, 1:16, 1:32, 1:64, 1:128 and 1:256 made into 1×PBS buffer were tested (in top to bottom order in FIG. 6).

### BRIEF DESCRIPTION OF THE SEQUENCES

Protein sequence of monoclonal antibody 2A4 heavy chain variable domain (VH)
complementarity determining region 1 (CDR1), (SEQ ID NO: 1)
GFTFSDAW
Protein sequence of monoclonal antibody 2A4 heavy chain variable domain (VH)
complementarity determining region 2 (CDR2), (SEQ ID NO: 2)
IRDETNNHIT
Protein sequence of monoclonal antibody 2A4 heavy chain variable domain (VH)
complementarity determining region 3 (CDR3), (SEQ ID NO: 3)
AAGFAY
Protein sequence of monoclonal antibody 2A4 light chain variable domain (VL)
complementarity determining region 1 (CDR1), (SEQ ID NO: 4)
QSLEKSNGKTY
Protein sequence of monoclonal antibody 2A4 light chain variable domain (VL)
complementarity determining region 2 (CDR2), (SEQ ID NO: 5)
RVS
Protein sequence of monoclonal antibody 2A4 light chain variable domain (VL)
complementarity determining region 3 (CDR3), (SEQ ID NO: 6)
LQVSHVPFT
Protein sequence of monoclonal antibody 2H7 heavy chain variable domain (VH)
complementarity determining region 1 (CDR1), (SEQ ID NO: 7)
GYSITSGYS
Protein sequence of monoclonal antibody 2H7 heavy chain variable domain (VH)
complementarity determining region 2 (CDR2), (SEQ ID NO: 8)
IHYSGRT
Protein sequence of monoclonal antibody 2H7 heavy chain variable domain (VH)
complementarity determining region 3 (CDR3), (SEQ ID NO: 9)
ARSMDY
Protein sequence of monoclonal antibody 2H7 light chain variable domain (VL)
complementarity determining region 1 (CDR1), (SEQ ID NO: 10)
QDIKTY
Protein sequence of monoclonal antibody 2H7 light chain variable domain (VL)
complementarity determining region 2 (CDR2), (SEQ ID NO: 11)
YAT
Protein sequence of monoclonal antibody 2H7 light chain variable domain (VL)
complementarity determining region 3 (CDR3), (SEQ ID NO: 12)
LQHGESPPT
Protein sequence of monoclonal antibody 9B8 heavy chain variable domain (VH)
complementarity determining region 1 (CDR1), (SEQ ID NO: 13)
GFTFSDAW
Protein sequence of monoclonal antibody 9B8 heavy chain variable domain (VH)
complementarity determining region 2 (CDR2), (SEQ ID NO: 14)
IRSKAKNHIT
Protein sequence of monoclonal antibody 9B8 heavy chain variable domain (VH)
complementarity determining region 3 (CDR3), (SEQ ID NO: 15)
TGGFAY
Protein sequence of monoclonal antibody 9B8 light chain variable domain (VL)
complementarity determining region 1 (CDR1), (SEQ ID NO: 16)
QSLEKSNGNTY
Protein sequence of monoclonal antibody 9B8 light chain variable domain (VL)
complementarity determining region 2 (CDR2), (SEQ ID NO: 17)
RVS
Protein sequence of monoclonal antibody 9B8 light chain variable domain (VL)
complementarity determining region 3 (CDR3), (SEQ ID NO: 18)
VQVSHVPFT
Protein sequence of monoclonal antibody 2A4 heavy chain variable domain (VH),
(SEQ ID NO: 19)
AVNLEESGGGLVLPGGSMKLSCTASGFTFSDAWMDWVRRS
PEKGLEWLAEIRDETNNHITYYAESVKVRFIISRDDSKSSVY
LQMNNLRPEDTGIYYCAAGFAYWGQGTLVTVSA
Protein sequence of monoclonal antibody 2A4 light chain variable domain (VL),
(SEQ ID NO: 20)
DAVMTQTPLSLSVSLGDQASISCRSSQSLEKSNGKTYLNWY
LQKPGQSPQLLIYRVSNRFSGVLDRFSGSGSGTDFTLKISRV
EAEDLGLYFCLQVSHVPFTFGSGTKLEIK
Protein sequence of monoclonal antibody 2H7 heavy chain variable domain (VH),
(SEQ ID NO: 21)
DVQLQESGPDLVKPSQSLSLTCTVTGYSITSGYSWHWIRQF
PGNKLEWMGYIHYSGRTNYNPSLKSRISITRDTSKNQFFLQ
LNSVTTEDTATYYCARSMDYWGQGTSVSVSS
Protein sequence of monoclonal antibody 2H7 light chain variable domain (VL),
(SEQ ID NO: 22)
DIKMTQSPSSMYASLGERVTITCKASQDIKTYLNWYQQKPW
KSPKTLIYYATSLADGVPSRFSGSGSGQDFSLTISSLESDDTA
TYFCLQHGESPPTFGGGTKLEIK
Protein sequence of monoclonal antibody 9B8 heavy chain variable domain (VH),
(SEQ ID NO: 23)
EVKLEESGGGLVQPGGSMKLSCAASGFTFSDAWMDWVRRS
PEKGLEWIAEIRSKAKNHITYYAESVKGRFTISRDDSKSSVY
LQMNSLRTEDSGIYYCTGGFAYWGQGTLVTVSA
Protein sequence of monoclonal antibody 9B8 light chain variable domain (VL),
(SEQ ID NO: 24)
DAVMTQNPLSLPVSLGDQASISCRSSQSLEKSNGNTYLNWY
LQKPGQSPQLLIYRVSNRFSGVPDRISGGGSGTDFTLKISRV
EAEDLGVYFCVQVSHVPFTFGSGTKLEIK
Nucleic acid sequence encoding monoclonal antibody 2A4 heavy chain variable do-
main (VH) complementarity determining region 1 (CDR1), (SEQ ID NO: 25)
ggattcactt ttagtgacgc ctgg
Nucleic acid sequence encoding monoclonal antibody 2A4 heavy chain variable do-
main (VH) complementarity determining region 2 (CDR2), (SEQ ID NO: 26)
attagagacg aaactaataa tcatataaca
Nucleic acid sequence encoding monoclonal antibody 2A4 heavy chain variable do-
main (VH) complementarity determining region 3 (CDR3), (SEQ ID NO: 27)
gcggctggat ttgcttac
Nucleic acid sequence encoding monoclonal antibody 2A4 light chain variable do-
main (VL) complementarity determining region 1 (CDR1), (SEQ ID NO: 28)
cagagccttg aaaagagtaa tggaaaaacc tat
Nucleic acid sequence encoding monoclonal antibody 2A4 light chain variable do-
main (VL) complementarity determining region 2 (CDR2), (SEQ ID NO: 29)
agggtttcc
Nucleic acid sequence encoding monoclonal antibody 2A4 light chain variable do-
main (VL) complementarity determining region 3 (CDR3), (SEQ ID NO: 30)
ctccaagttt cacatgtccc attcacg
Nucleic acid sequence encoding monoclonal antibody 2H7 heavy chain variable do-
main (VH) complementarity determining region 1 (CDR1), (SEQ ID NO: 31)
ggctactcca tcaccagtgg ttatagc
Nucleic acid sequence encoding monoclonal antibody 2H7 heavy chain variable do-
main (VH) complementarity determining region 2 (CDR2), (SEQ ID NO: 32)
atacactaca gtggtcgcac t
Nucleic acid sequence encoding monoclonal antibody 2H7 heavy chain variable do-
main (VH) complementarity determining region 3 (CDR3), (SEQ ID NO: 33)
gcaagatcta tggactac
Nucleic acid sequence encoding monoclonal antibody 2H7 light chain variable do-
main (VL) complementarity determining region 1 (CDR1), (SEQ ID NO: 34)
caggacatta aaacctat
Nucleic acid sequence encoding monoclonal antibody 2H7 light chain variable do-
main (VL) complementarity determining region 2 (CDR2), (SEQ ID NO: 35)
tatgcaaca
Nucleic acid sequence encoding monoclonal antibody 2H7 light chain variable do-
main (VL) complementarity determining region 3 (CDR3), (SEQ ID NO: 36)
ctacagcatg gtgagagccc toccacg
Nucleic acid sequence encoding monoclonal antibody 9B8 heavy chain variable do-
main (VH) complementarity determining region 1 (CDR1), (SEQ ID NO: 37)
ggattcactt ttagtgacgc ctgg
Nucleic acid sequence encoding monoclonal antibody 9B8 heavy chain variable do-
main (VH) complementarity determining region 2 (CDR2), (SEQ ID NO: 38)
attagaagca aagctaaaaa tcacataaca
Nucleic acid sequence encoding monoclonal antibody 9B8 heavy chain variable do-
main (VH) complementarity determining region 3 (CDR3), (SEQ ID NO: 39)
acaggggggt ttgcttac
Nucleic acid sequence encoding monoclonal antibody 9B8 light chain variable do-
main (VL) complementarity determining region 1 (CDR1), (SEQ ID NO: 40)
cagagtcttg aaaaaagtaa tggaaacacc tat
Nucleic acid sequence encoding monoclonal antibody 9B8 light chain variable do-
main (VL) complementarity determining region 2 (CDR2), (SEQ ID NO: 41)
agggtttcc
Nucleic acid sequence encoding monoclonal antibody 9B8 light chain variable do-
main (VL) complementarity determining region 3 (CDR3), (SEQ ID NO: 42)
gtccaagttt cacatgtccc attcacg
Nucleic acid sequence encoding monoclonal antibody 2A4 heavy chain variable do-
main (VH), (SEQ ID NO: 43)

| | |
|---|---|
| gcagtgaatc ttgaggagtc tggaggaggc ttggtgctac ctggaggatc catgaaactc | 60 |
| tcttgtactg cctctggatt cacttttagt gacgcctgga tggactgggt ccgccggtct | 120 |
| ccagagaagg ggcttgagtg gcttgctgaa attagagacg aaactaataa tcatataaca | 180 |
| tattatgctg agtctgtgaa agtgaggttc atcatctcaa gagatgattc caaaagtagt | 240 |
| gtctaccttc aaatgaacaa cttaagacct gaagacactg gcatttatta ctgtgcggct | 300 |
| ggatttgctt actggggcca agggactctg gtcactgtct ctgca | 345 |

Nucleic acid sequence encoding monoclonal antibody 2A4 light chain variable do-
main (VL), (SEQ ID NO: 44)

| | |
|---|---|
| gatgctgtga tgacccaaac tccactctcc ctgtctgtca gtcttggaga tcaagcctcc | 60 |
| atctcttgca ggtctagtca gagccttgaa aagagtaatg gaaaaaccta tttgaactgg | 120 |
| tacctccaga aaccaggcca gtctccacag ctcctgatct atagggtttc caaccgattt | 180 |
| tctggggtcc tagacaggtt cagtggtagt ggatcaggga cagatttcac actgaaaatc | 240 |
| agtagagtgg aggctgagga tttgggactt tatttctgcc tccaagtttc acatgtccca | 300 |
| ttcacgttcg gctcggggac aaagttggaa ataaaa | 336 |

Nucleic acid sequence encoding monoclonal antibody 2H7 heavy chain variable do-
main (VH), (SEQ ID NO: 45)

| | |
|---|---|
| gatgtgcagc ttcaggagtc aggacctgac ctggtgaaac cttctcagtc actttcactc | 60 |
| acctgcactg tcactggcta ctccatcacc agtggttata gctggcactg gatccggcag | 120 |
| tttccaggaa acaaactgga atggatgggc tatatacact acagtggtcg cactaactac | 180 |
| aacccatctc tcaaaagtcg aatctctatc actcgagaca catccaagaa ccagttcttc | 240 |
| ctgcagttga attctgtgac tactgaggac acagccacat attactgtgc aagatctatg | 300 |
| gactactggg gtcaaggaac ctcagtctcc gtctcctca | 339 |

Nucleic acid sequence encoding monoclonal antibody 2H7 light chain variable do-
main (VL), (SEQ ID NO: 46)

| | |
|---|---|
| gacatcaaga tgacccagtc tccatcctcc atgtatgcat cgctgggaga gagagtcact | 60 |
| atcacttgca aggcgagtca ggacattaaa acctatttaa actggtacca gcagaaacca | 120 |
| tggaaatctc ctaagaccct gatctattat gcaacaagct tggcagatgg ggtcccatca | 180 |
| cgattcagtg gcagtggatc tggacaagat ttttctctaa ccatcagcag cctggagtct | 240 |
| gacgatacag caacttattt ctgtctacag catggtgaga gccctcccac gttcggaggg | 300 |
| gggaccaaac tggaaataaa a | 321 |

Nucleic acid sequence encoding monoclonal antibody 9B8 heavy chain variable do-
main (VH), (SEQ ID NO: 47)

| | |
|---|---|
| gaagtgaagc ttgaggagtc tggaggaggc ttggtgcaac ctggaggatc catgaaactc | 60 |
| tcttgtgctg cctctggatt cacttttagt gacgcctgga tggactgggt ccgccggtct | 120 |
| ccagagaagg ggcttgagtg gattgctgaa attagaagca aagctaaaaa tcacataaca | 180 |
| tactatgctg agtctgtgaa ggggaggttc accatctcaa gagatgattc caaaagtagt | 240 |
| gtctacctac aaatgaacag cttaagaact gaagactctg gcatttatta ctgtacaggg | 300 |
| gggtttgctt actggggcca agggactctg gtcactgtct ctgca | 345 |

Nucleic acid sequence encoding monoclonal antibody 9B8 light chain variable do-
main (VL), (SEQ ID NO: 48)

| | |
|---|---|
| gatgctgtga tgacccaaaa tccactctcc ctgcctgtca gtcttggaga tcaagcctcc | 60 |
| atctcttgca ggtctagtca gagtcttgaa aaaagtaatg gaaacaccta tttgaactgg | 120 |
| tacctccaga aaccaggcca gtctccacag ctcctgatat acagggtttc caaccgattt | 180 |
| tctggggtcc cagacaggat cagtggtggt ggatcaggga cagatttcac actgaaaatc | 240 |
| agcagagtgg aggctgagga tttgggagtt tatttctgcg tccaagtttc acatgtccca | 300 |
| ttcacgttcg gctcggggac aaagttggaa ataaaa | 336 |

### DETAILED DESCRIPTION OF THE INVENTION

Relaxin is a protein hormone that is released from both the placenta and in many species, also from the ovaries. It is primarily secreted from the placenta in cats and dogs, making it a useful test in pregnancy diagnosis. Relaxin has a role in softening of the cervix around the time of parturition to relax this otherwise firm structure to permit delivery. Circulating concentrations of placental relaxin are elevated from approximately 21-24 d after the luteinizing hormone (LH) surge to the end of pregnancy. Relaxin can be detected in the blood in most pregnant female dogs as early as 22-27 days post-breeding (post-ovulation). The level of relaxin remains elevated throughout pregnancy and declines rapidly following the end of the pregnancy. In pregnant canine bitches relaxin reaches peak concentrations of 5 ng/ml to 50 ng/ml in late pregnancy (40-50 d of gestation). During pseudopregnancy relaxin is not produced. Thus, relaxin can be used to discriminate between pregnancy and pseudopregnancy.

Relaxin is a 6 kDa peptide hormone belonging to insulin like growth factor family. Chemical synthesis or expression of relaxin hormone in an active form may be challenging as it is synthesized as a single-chain precursor, preprorelaxin that is processed by cleavage of the signal peptide and by internal cleavage of a connecting peptide (C domain peptide) to form a heterodimer of two disulfidelinked chains, the A chain and the B chain, which form the active hormone.

In pregnant feline queens, relaxin is produced by trophoblast cells of the lamellar placental labyrinth and its primary functions are thought to be related to gestation and parturition. Implantation of the embryo in the uterus occurs between days 12 and 13 of gestation and is followed by a surge in circulating relaxin concentrations between days 20 and 35. Relaxin is also believed to work synergistically with progesterone in uterine tissue to maintain pregnancy. Relaxin production continues until parturition and contributes to the softening of fibrous connective tissues of the interpubic ligament, a change that facilitates delivery of fetuses through the birth canal. Relaxin concentrations generally return to undetectable levels within 24 hours of parturition and are not detectable during the estrous cycle or during pseudopregnancy in cats, which optimizes its value as an aid in diagnosis of pregnancy in this species.

In the context of the present application, the term “antibody” is used as a synonym for “immunoglobulin” (Ig), which is defined as a protein belonging to the class IgG, IgM, IgE, IgA, or IgD (or any subclass thereof), and includes all conventionally known antibodies and functional fragments thereof. Typically, an antibody consists of four polypeptide chains; two heavy chains and two light chains. Light chains consist of one variable domain V_{L} and one constant domain C_{L}, while heavy chains contain one variable domain VH and three to four constant domains C_{H}1, C_{H}2 etc. The V_{L} domain comprises a CDR1 region (CDRL1), a CDR2 region (CDRL2), a CDR3 region (CDRL3) and Framework (FR) regions. The V_{H} domain comprises a CDR1 region (CDRH1), a CDR2 region (CDRH2), a CDR3 region (CDRH3) and Framework regions.

In the context of the present invention, a “functional fragment” of an antibody/immunoglobulin is defined as a derivative of a parental antibody that essentially maintains one or more of the properties of the parental antibody, particularly the ability to recognize and bind to the same epitope. The functional fragment described herein is characterized by a specific percentage identity to the parent antibody, and its ability to still bind to relaxin, preferably with at least the same affinity than the parent antibody.

As used herein, the percent homology between two amino acid sequences is equivalent to the percent identity between the two sequences. The percent identity between the two sequences is a function of the number of identical positions shared by the sequences (i.e. % homology=# of identical positions/total # of positions×100), taking into account the number of gaps, and the length of each gap, which need to be introduced for optimal alignment of the two sequences. The comparison of sequences and determination of percent identity between two sequences can be accomplished using standard methods known in the art.

Specifically, sequence identity can be determined using the NCBI BLAST-program package using the pre-set parameters, wherein the sequence identity needs to be calculated at best fit over the whole length of the sequence of the present disclosure.

Binding affinities can be determined using routine methods known to the person skilled in the art. In preferred functional fragments is the binding affinity of the functional fragment at least as high as the binding affinity of the parent antibody. Accordingly, one example of a functional fragment is an affinity matured antibody.

The functional fragment is a fragment (e.g., a variable region of an IgG) that retains the antigen-binding region. An “antigen-binding region” of an antibody typically is found in one or more hypervariable region(s) of an antibody, i.e., the CDR1, CDR2, and/or CDR3 regions. Functional fragments of antibodies include but are not limited to fragments such as Fab, Fab′, F(ab′)₂ or Fv fragment; a light chain or heavy chain monomer or dimer; or a single chain antibody, e.g. a single chain Fv (scFv) in which heavy and light chain variable regions are joined by a peptide linker; a dimerized V region fragment (diabody), a disulfide stabilized V region fragment (ds), triabodies, tetrabodies, Fc fusion proteins, peptides containing CDR and the like or any other recombinant, or CDR-grafted molecule. Similarly, the heavy and light chain variable region may be combined with other antibody domains as appropriate. The F(ab′)₂ or Fab may be engineered to minimize or completely remove the intermolecular disulphide interactions that occur between the C_{H}1 and C_{L} domains. The antibodies or functional fragments may be part of bi- or multifunctional constructs. Recombinant antibodies or functional fragments such as chimeric, primatized, humanized, or human antibodies may also be used. Different recombinant methodologies are available to one of ordinary skill in the art to produce such antibodies or functional fragments.

The present invention provides an in vitro assay method for relaxin, comprising capturing and detecting relaxin. Specifically, the assay method for relaxin comprises
(a) binding relaxin with a capture antibody comprising
a VL domain comprising a CDR1 region having an amino acid sequence set forth in SEQ ID NO: 10, a CDR2 region having an amino acid sequence set forth in SEQ ID NO: 11, and a CDR3 region having an amino acid sequence set forth in SEQ ID NO: 12, and a VH domain comprising a CDR1 region having an amino acid sequence set forth in SEQ ID NO: 7, a CDR2 region having an amino acid sequence set forth in SEQ ID NO: 8, and a CDR3 region having an amino acid sequence set forth in SEQ ID NO: 9;
or a functional fragment thereof which has independently at least 80% sequence identity, or at least 85%, 90%, 95%, 96%, 97%, 98% or 99% sequence identity to any one of SEQ ID NO: 7-12, wherein the functional fragment is capable of binding relaxin, in particular canine or feline relaxin, preferably canine relaxin;
and
(b) binding relaxin with a first detection antibody comprising
a VL domain comprising a CDR1 region having an amino acid sequence set forth in SEQ ID NO: 4, a CDR2 region having an amino acid sequence set forth in SEQ ID NO: 5, and a CDR3 region having an amino acid sequence set forth in SEQ ID NO: 6, and a VH domain comprising a CDR1 region having an amino acid sequence set forth in SEQ ID NO: 1, a CDR2 region having an amino acid sequence set forth in SEQ ID NO: 2, and a CDR3 region having an amino acid sequence set forth in SEQ ID NO: 3;
or a functional fragment thereof which has independently at least 80% sequence identity, or at least 85%, 90%, 95%, 96%, 97%, 98% or 99% sequence identity to any one of SEQ ID NO: 1-6, wherein the functional fragment is capable of binding relaxin, in particular canine or feline relaxin, preferably canine relaxin;
and
(c) binding relaxin with a second detection antibody comprising
a VL domain comprising a CDR1 region having an amino acid sequence set forth in SEQ ID NO: 16, a CDR2 region having an amino acid sequence set forth in SEQ ID NO: 17, and a CDR3 region having an amino acid sequence set forth in SEQ ID NO: 18, and a VH domain comprising a CDR1 region having an amino acid sequence set forth in SEQ ID NO: 13, a CDR2 region having an amino acid sequence set forth in SEQ ID NO: 14, and a CDR3 region having an amino acid sequence set forth in SEQ ID NO: 15;
or a functional fragment thereof which has independently at least 80% sequence identity, or at least 85%, 90%, 95%, 96%, 97%, 98% or 99% sequence identity to any one of SEQ ID NO: 13-18, wherein the functional fragment is capable of binding relaxin, in particular canine or feline relaxin, preferably canine relaxin.

In another aspect, the assay method for relaxin comprises
(a) binding relaxin with a capture antibody comprising
a VH domain having an amino acid sequence set forth in SEQ ID NO: 21, and a VL domain having an amino acid sequence set forth in SEQ ID NO: 22;
or a functional fragment thereof which has independently at least 80% sequence identity, or at least 85%, 90%, 95%, 96%, 97%, 98% or 99% sequence identity to any one of SEQ ID NO: 21 or 22, wherein the functional fragment is capable of binding relaxin, in particular canine or feline relaxin, preferably canine relaxin;
and
(b) binding relaxin with a first detection antibody comprising
a VH domain having an amino acid sequence set forth in SEQ ID NO: 19, and a VL domain having an amino acid sequence set forth in SEQ ID NO: 20;
or a functional fragment thereof which has independently at least 80% sequence identity, or at least 85%, 90%, 95%, 96%, 97%, 98% or 99% sequence identity to any one of SEQ ID NO: 19 or 20, wherein the functional fragment is capable of binding relaxin, in particular canine or feline relaxin, preferably canine relaxin;
and
(c) binding relaxin with a second detection antibody comprising
a VH domain having an amino acid sequence set forth in SEQ ID NO: 23, and a VL domain having an amino acid sequence set forth in SEQ ID NO: 24;
or a functional fragment thereof which has independently at least 80% sequence identity, or at least 85%, 90%, 95%, 96%, 97%, 98% or 99% sequence identity to any one of SEQ ID NO: 23 or 24, wherein the functional fragment is capable of binding relaxin, in particular canine or feline relaxin, preferably canine relaxin.

The relaxin detected by the method may be canine or feline relaxin, preferably canine relaxin. The assay may be an immunoassay such as ELISA, a Western blot, an immunohistochemical assay, or a mass spectrometric assay.

An “immunoassay” as used herein refers to a biochemical test that measures the presence or concentration of a certain molecule (also called analyte or antigen) in a sample through the use of an antibody. The immunoassay may be a competitive immunoassay where the analyte in a sample competes with a labelled analyte to bind the antibody, or the immunoassay may be a noncompetitive immunoassay where the analyte in a sample binds to a labelled antibody. In a two-site noncompetitive immunoassay a capture antibody may be used to capture the analyte from the sample to facilitate detection of the analyte. The capture antibody is typically bound to a surface so sample molecules other than the analyte may be washed away. Detection of the bound analyte may be performed using another antibody that typically binds to a different epitope on the analyte than the capture antibody or to a different copy of the same epitope in case the epitope is present as repeats on the same antigen molecule. This detection antibody may be labelled to allow for detection of bound detection antibody and through this also the analyte.

An antibody or functional fragment that binds relaxin is an anti-relaxin antibody. Preferably, the antibody or functional fragment specifically binds relaxin. The relaxin may be canine relaxin or feline relaxin i.e. the antibody or functional fragment may bind relaxin of both species or just one of them. Preferably, the relaxin is canine relaxin. As described in the example herein, immunization of mice to produce the antibodies of the invention was performed with preprorelaxin. It follows that some of the antibodies produced by the obtained clones may bind either the signal peptide or C peptide which are normally cleaved during relaxin maturation and are thus not part of the mature relaxin hormone present in the bloodstream of the pregnant females. The clones obtained from the immunizations were, however, screened for relaxin specificity using serum of a pregnant female dog, and so for example antibodies binding only those parts of preprorelaxin that are not present in the mature relaxin were excluded from further testing. The antibodies were further tested for their performance in relaxin testing in dog whole blood and serum by using the antibodies either as capture antibody or labelled detection antibody in an immunoassay performed in a strip test format.

As used herein, an antibody or functional fragment thereof “specifically recognizes”, or “specifically binds to” canine relaxin, when the antibody or functional fragment is able to discriminate between canine relaxin and one or more reference molecule(s) which may be non-relaxin molecules. In its most general form, “specific binding” is referring to the ability of the antibody or functional fragment to discriminate between relaxin and an unrelated biomolecule, as determined, for example, in accordance with any specificity assay method known in the art. Such methods comprise Western blots and ELISA tests. For example, a standard ELISA assay can be carried out.

The antibody can be an immunoglobulin, preferably an immunoglobulin G (IgG). The subclass of the antibody is not limited and includes IgG₁, IgG₂, IgG₃, and IgG₄.

The antibody used in the present method is a monoclonal antibody. The term “monoclonal antibody” as used herein is not limited to antibodies produced through hybridoma technology. The term “monoclonal antibody” refers to an antibody that is derived from a clone of a single B-cell lineage, including any eukaryotic, prokaryotic, or phage clone, and not the method by which it is produced. Monoclonal antibodies can be prepared using a wide variety of techniques known in the art including the use of hybridoma, recombinant, and phage display technologies, or a combination thereof.

The term “polyclonal antibody” as used herein refers to antibody that is derived from an immunization of a live animal. After immunization, polyclonal antibodies can be obtained or purified from the blood or serum of the animal. A disadvantage of polyclonal antibodies is their batch-to-batch variability as they are produced in different animals at different times. Also, there is a high chance of cross-reactivity due to a recognition of multiple epitopes as compared to monoclonal antibodies which typically only recognize a single epitope.

All antibodies, functional fragments thereof and nucleic acid molecules are preferably isolated antibodies, isolated functional fragments thereof and isolated nucleic acid molecules.

Of the total of 15 isolated monoclonal antibodies tested for their performance in the relaxin immunoassay, the performance of antibodies 2H7, 2A4 and 9B8 exceeded that of the other antibodies. In particular, 2H7 was considered to perform well as a capture antibody, and 2A4 and 9B8 as detection antibodies either alone or as a combination. As disclosed above, antibody 2A4 is characterised by comprising a V_{L} CDR1, CDR2 and CDR3 set forth in SEQ ID NOs: 4, 5 and 6, respectively, and V_{H} CDR1, CDR2 and CDR3 set forth in SEQ ID NOs: 1, 2 and 3, respectively. Further, as disclosed above, antibody 2H7 is characterised by comprising a V_{L} CDR1, CDR2 and CDR3 set forth in SEQ ID NOs: 10, 11 and 12, respectively, and V_{H} CDR1, CDR2 and CDR3 set forth in SEQ ID NOs: 7, 8 and 9, respectively. Further, as disclosed above, antibody 9B8 is characterised by comprising a V_{L} CDR1, CDR2 and CDR3 set forth in SEQ ID NOs: 16, 17 and 18, respectively, and V_{H} CDR1, CDR2 and CDR3 set forth in SEQ ID NOs: 13, 14 and 15, respectively.

Further, as disclosed above, antibody 2A4 is characterised by comprising a V_{H} domain set forth in SEQ ID NO: 19, and a V_{L} domain set forth in SEQ ID NO: 20. Further, as disclosed above, antibody 2H7 is characterised by comprising a V_{H} domain set forth in SEQ ID NO: 21, and a V_{L} domain set forth in SEQ ID NO: 22. Further, as disclosed above, antibody 9B8 is characterised by comprising a V_{H} domain set forth in SEQ ID NO: 23, and a V_{L} domain set forth in SEQ ID NO: 24.

Usually, the method comprises forming a sandwich between relaxin, a capture antibody and a detection antibody. In one embodiment, the method comprises forming a sandwich between relaxin, a capture antibody and a detection antibody, wherein the capture antibody is 2H7 or functional fragment thereof and the detection antibody is 2A4 or functional fragment thereof and 9B8 or functional fragment thereof.

The good performance of these antibodies in the relaxin assay of the present invention also render these antibodies valuable as such.

Accordingly, the disclosure further provides an anti-relaxin antibody or a functional fragment thereof, comprising a V_{L} domain comprising a CDR1 region having an amino acid sequence set forth in SEQ ID NO: 4; a CDR2 region having an amino acid sequence set forth in SEQ ID NO: 5; and a CDR3 region having an amino acid sequence set forth in SEQ ID NO: 6; and a V_{H} domain comprising a CDR1 region having an amino acid sequence set forth in SEQ ID NO: 1; a CDR2 region having an amino acid sequence set forth in SEQ ID NO: 2; and a CDR3 region having an amino acid sequence set forth in SEQ ID NO: 3.

The disclosure also provides an anti-relaxin antibody or a functional fragment thereof, comprising a V_{L} domain comprising a CDR1 region having an amino acid sequence set forth in SEQ ID NO: 10; a CDR2 region having an amino acid sequence set forth in SEQ ID NO: 11; and a CDR3 region having an amino acid sequence set forth in SEQ ID NO: 12; and a V_{H} domain comprising a CDR1 region having an amino acid sequence set forth in SEQ ID NO: 7; a CDR2 region having an amino acid sequence set forth in SEQ ID NO: 8; and a CDR3 region having an amino acid sequence set forth in SEQ ID NO: 9.

The disclosure additionally provides an anti-relaxin antibody or a functional fragment thereof, comprising a V_{L} domain comprising a CDR1 region having an amino acid sequence set forth in SEQ ID NO: 16; a CDR2 region having an amino acid sequence set forth in SEQ ID NO: 17; and a CDR3 region having an amino acid sequence set forth in SEQ ID NO: 18; and a V_{H} domain comprising a CDR1 region having an amino acid sequence set forth in SEQ ID NO: 13; a CDR2 region having an amino acid sequence set forth in SEQ ID NO: 14; and a CDR3 region having an amino acid sequence set forth in SEQ ID NO: 15.

The invention further provides an anti-relaxin antibody or functional fragment thereof, comprising a V_{H} domain having an amino acid sequence set forth in SEQ ID NO: 19, and a V_{L} domain having an amino acid sequence set forth in SEQ ID NO: 20.

The invention also provides an anti-relaxin antibody or functional fragment thereof, comprising a V_{H} domain having an amino acid sequence set forth in SEQ ID NO: 21, and a V_{L} domain having an amino acid sequence set forth in SEQ ID NO: 22.

The invention provides an anti-relaxin antibody or functional fragment thereof, comprising a V_{H} domain having an amino acid sequence set forth in SEQ ID NO: 23, and a V_{L} domain having an amino acid sequence set forth in SEQ ID NO: 24.

The polypeptides and polynucleotides according to the present embodiments include those, which have at least 80% sequence identity, or at least 85%, 90%, 95%, 96%, 97%, 98% or 99% sequence identity to the anti-relaxin antibodies or functional fragments thereof or to the polynucleotides encoding said antibodies or functional fragments. In a further embodiment, the at least 80% sequence identity, or at least 85%, 90%, 95%, 96%, 97%, 98% or 99% sequence identity, is outside the sequence region defining the V_{L} and VH CDR1, CDR2 and CDR3 regions described herein. That is, within the CDR regions sequence identity is 100%.

In an embodiment, an isolated monoclonal anti-relaxin antibody or functional fragment thereof comprises a V_{H} domain and a V_{L} domain, wherein: (a) the V_{H} domain comprises an amino acid sequence that has at least 80% sequence identity or at least 85%, 90%, 95%, 96%, 97%, 98% or 99% sequence identity to an amino acid sequence selected from the group consisting of SEQ ID NOs: 19, 21 or 23; (b) the V_{L} domain comprises an amino acid sequence that has at least 80% sequence identity or at least 85%, 90%, 95%, 96%, 97%, 98% or 99% sequence identity to an amino acid sequence selected from the group consisting of SEQ ID NOs: 20, 22 or 24. In a further embodiment, the at least 80% sequence identity, or at least 85%, 90%, 95%, 96%, 97%, 98% or 99% sequence identity, is outside the sequence region defining the V_{L} and VH CDR1, CDR2 and CDR3 regions described herein. That is, within the CDR regions sequence identity is 100%.

The anti-relaxin antibody or functional fragment thereof can further comprise heavy and light chain variable regions and/or CDRs comprising amino acid sequences that are homologous to the amino acid sequences of the antibodies described herein, and wherein the antibodies retain the desired functional properties of the anti-relaxin antibodies or functional fragments thereof according to the various embodiments of the invention. In some further embodiments, an antibody or functional fragment thereof capable of binding to relaxin binds to essentially the same epitope as the antibody or functional fragment thereof according to this invention.

Accordingly, the VH and/or V_{L} amino acid sequences may be at least 80% or at least 85%, 90%, 95%, 96%, 97%, 98% or 99% homologous to the sequences set forth above. An antibody having VH and V_{L} regions having high (i.e., 80% or greater) homology to the V_{H} and V_{L} regions of the sequences set forth above, can be obtained by mutagenesis (e.g., site-directed or PCR-mediated mutagenesis) of nucleic acid molecules encoding SEQ ID NO:s 19, 21 or 23 and 20, 22 or 24, followed by testing of the encoded altered antibody for retained function. In a further embodiment, the at least 80% sequence homology, or at least 85%, 90%, 95%, 96%, 97%, 98% or 99% sequence homology, is outside the sequence region defining the V_{L} and V_{H} CDR1, CDR2 and CDR3 regions described herein. That is, within the CDR regions sequence homology is 100%.

It is well known in the art that the CDR3 domain, independently from the CDR1 and/or CDR2 domain(s), alone can determine the binding specificity of an antibody for a cognate antigen and that multiple antibodies can predictably be generated having the same binding specificity based on a common CDR3 sequence. Accordingly, the present disclosure provides monoclonal anti-relaxin antibodies and functional fragments thereof comprising one or more heavy and/or light chain CDR3 domain(s) as disclosed herein. Within some embodiments, such antibodies comprising one or more heavy and/or light chain CDR3 domain(s) as disclosed herein (a) are capable of competing for binding with; (b) retain the functional characteristics; (c) bind to the same epitope; and/or (d) have a similar binding affinity as the corresponding parental antibody.

Accordingly, the disclosure provides an anti-relaxin antibody or a functional fragment thereof, comprising a V_{L} domain comprising a CDR1 region having an amino acid sequence selected from the group consisting of sequences set forth in SEQ ID NOs: 4, 10 or 16; a CDR2 region having an amino acid sequence selected from the group consisting of sequences set forth in SEQ ID NOs: 5, 11 or 17; and a CDR3 region having an amino acid sequence selected from the group consisting of sequences set forth in SEQ ID NOs: 6, 12 or 18, and a V_{H} domain comprising a CDR1 region having an amino acid sequence selected from the group consisting of sequences set forth in SEQ ID NOs: 1, 7 or 13; a CDR2 region having an amino acid sequence selected from the group consisting of sequences set forth in SEQ ID NOs: 2, 8 or 14; and a CDR3 region having an amino acid sequence selected from the group consisting of sequences set forth in SEQ ID NOs: 3, 9 or 15. The antibody is preferably an isolated monoclonal antibody.

Further contemplated is an anti-relaxin antibody or a functional fragment thereof, comprising a V_{L} domain comprising a CDR3 region having an amino acid sequence set forth in SEQ ID NO: 6, and a CDR1 region having an amino acid sequence selected from the group consisting of sequences set forth in SEQ ID NOs: 4, 10 or 16; a CDR2 region having an amino acid sequence selected from the group consisting of sequences set forth in SEQ ID NOs: 5, 11 or 17; and a V_{H} domain comprising a CDR3 region having an amino acid sequence set forth in SEQ ID NO: 3, and a CDR1 region having an amino acid sequence selected from the group consisting of sequences set forth in SEQ ID NOs: 1, 7 or 13; a CDR2 region having an amino acid sequence selected from the group consisting of sequences set forth in SEQ ID NOs: 2, 8 or 14, or
a V_{L} domain comprising a CDR3 region having an amino acid sequence set forth in SEQ ID NO: 12, and a CDR1 region having an amino acid sequence selected from the group consisting of sequences set forth in SEQ ID NOs: 4, 10 or 16; a CDR2 region having an amino acid sequence selected from the group consisting of sequences set forth in SEQ ID NOs: 5, 11 or 17; and a V_{H} domain comprising a CDR3 region having an amino acid sequence set forth in SEQ ID NO: 9, and a CDR1 region having an amino acid sequence selected from the group consisting of sequences set forth in SEQ ID NOs: 1, 7 or 13; a CDR2 region having an amino acid sequence selected from the group consisting of sequences set forth in SEQ ID NOs: 2, 8 or 14, or
a V_{L} domain comprising a CDR3 region having an amino acid sequence set forth in SEQ ID NO: 18, and a CDR1 region having an amino acid sequence selected from the group consisting of sequences set forth in SEQ ID NOs: 4, 10 or 16; a CDR2 region having an amino acid sequence selected from the group consisting of sequences set forth in SEQ ID NOs: 5, 11 or 17; and a V_{H} domain comprising a CDR3 region having an amino acid sequence set forth in SEQ ID NO: 15, and a CDR1 region having an amino acid sequence selected from the group consisting of sequences set forth in SEQ ID NOs: 1, 7 or 13; a CDR2 region having an amino acid sequence selected from the group consisting of sequences set forth in SEQ ID NOs: 2, 8 or 14.

Further provided is an anti-relaxin antibody or functional fragment thereof, comprising a V_{H} domain having an amino acid sequence selected from the group consisting of sequences set forth in SEQ ID NOs: 19, 21 or 23, and a V_{L} domain having an amino acid sequence selected from the group consisting of sequences set forth in SEQ ID NOs: 20, 22 or 24. The antibody is preferably an isolated monoclonal antibody.

Further provided is an isolated monoclonal anti-relaxin antibody or functional fragment thereof, comprising a V_{H} domain and a V_{L} domain, wherein: (a) the V_{H} domain comprises an amino acid sequence that is at least 80% homologous to an amino acid sequence selected from the group consisting of SEQ ID NOs: 19, 21 or 23; and (b) the V_{L} domain comprises an amino acid sequence that is at least 80% homologous to an amino acid sequence selected from the group consisting of SEQ ID NOs: 20, 22 or 24. Optionally, the at least 80% sequence homology is outside the sequence region defining the V_{L} and V_{H} CDR1, CDR2 and CDR3 regions described herein. That is, within the CDR regions sequence homology is 100%.

As used herein, an anti-relaxin antibody or functional fragment thereof refers to an antibody or functional fragment thereof that is capable of binding to relaxin, preferably canine or feline relaxin, more preferably canine relaxin.

The assay can use at least one of the antibodies or functional fragments thereof of the present invention as capture and/or detection antibody. In the present invention, the capture antibody is 2H7 or functional fragment thereof and the detection antibody is 2A4 or functional fragment thereof and 9B8 or functional fragment thereof.

Further provided is a kit for pregnancy testing in an animal, wherein the kit comprises at least one of the capture antibodies and the detection antibodies or functional fragments thereof as defined in the preceding method, optionally wherein the animal is a dog or a cat.

The present invention provides a kit for pregnancy testing in a dog and/or a cat, wherein the kit comprises as a binding antibody or detection antibody at least one of the antibodies or functional fragments thereof of the present disclosure. In some embodiments, the at least one antibody or functional fragment thereof may comprise a detectable label. A person skilled in the art can readily determine any further reagents to be included in the kit depending on the desired technique for carrying out pregnancy testing in a dog and/or a cat. Thus, the kit may further comprise at least one reagent for performing for example an immunoassay such as ELISA, a Western blot, an immunohistochemical assay, or a mass spectrometric assay. In some embodiments, the kit may further comprise instructions for using the kit.

In one embodiment, the kit comprises at least one binding body selected from a group consisting of antibody 2H7 or functional fragment thereof, antibody 2A4 or functional fragment thereof and 9B8 or functional fragment thereof. Preferably, the kit comprises as capture antibody 2H7 or functional fragment thereof and as detection antibody 2A4 or functional fragment thereof and/or 9B8 or functional fragment thereof. Most preferably, the kit comprises antibody 2H7 or functional fragment thereof, antibody 2A4 or functional fragment thereof and 9B8 or functional fragment thereof.

Nucleic acid encoding the anti-relaxin antibody or functional fragment thereof according to the present disclosure are also provided. As disclosed above, antibody 2A4 is characterised by comprising a V_{L} CDR1, CDR2 and CDR3 encoded by nucleic acid of SEQ ID NOs: 28, 29 and 30, respectively, and V_{H} CDR1, CDR2 and CDR3 encoded by nucleic acid of SEQ ID NOs: 25, 26 and 27, respectively. Further, as disclosed above, antibody 2H7 is characterised by comprising a V_{L} CDR1, CDR2 and CDR3 encoded by nucleic acid of SEQ ID NOs: 34, 35 and 36, respectively, and V_{H} CDR1, CDR2 and CDR3 encoded by nucleic acid of SEQ ID NOs: 31, 32 and 33, respectively. Further, as disclosed above, antibody 9B8 is characterised by comprising a V_{L} CDR1, CDR2 and CDR3 encoded by nucleic acid of SEQ ID NOs: 40, 41 and 42, respectively, and V_{H} CDR1, CDR2 and CDR3 encoded by nucleic acid of SEQ ID NOs: 37, 38 and 39, respectively.

As disclosed above, antibody 2A4 is characterised by comprising a V_{H} domain encoded by nucleic acid of SEQ ID NO: 43, and a V_{L} domain encoded by nucleic acid of SEQ ID NO: 44. Further, as disclosed above, antibody 2H7 is characterised by comprising a V_{H} domain encoded by nucleic acid of SEQ ID NO: 45, and a V_{L} domain encoded by nucleic acid of SEQ ID NO: 46. Further, as disclosed above, antibody 9B8 is characterised by comprising a V_{H} domain encoded by nucleic acid of SEQ ID NO: 47, and a V_{L} domain encoded by nucleic acid of SEQ ID NO: 48.

Accordingly, the disclosure provides a nucleic acid encoding an anti-relaxin antibody or functional fragment thereof, comprising a V_{L} domain comprising a CDR1 region encoded by a nucleic acid sequence set forth in SEQ ID NO: 28; a CDR2 region encoded by a nucleic acid sequence set forth in SEQ ID NO: 29; and a CDR3 region encoded by a nucleic acid sequence set forth in SEQ ID NO: 30; and a V_{H} domain comprising a CDR1 region encoded by a nucleic acid sequence set forth in SEQ ID NO: 25; a CDR2 region encoded by a nucleic acid sequence set forth in SEQ ID NO: 26; and a CDR3 region encoded by a nucleic acid sequence set forth in SEQ ID NO: 27. The nucleic acid is preferably an isolated nucleic acid. Optionally, the nucleic acid is cDNA.

Furthermore, the disclosure provides a nucleic acid encoding an anti-relaxin antibody or functional fragment thereof, comprising a V_{L} domain comprising a CDR1 region encoded by a nucleic acid sequence set forth in SEQ ID NO: 34; a CDR2 region encoded by a nucleic acid sequence set forth in SEQ ID NO: 35; and a CDR3 region encoded by a nucleic acid sequence set forth in SEQ ID NO: 36; and a V_{H} domain comprising a CDR1 region encoded by a nucleic acid sequence set forth in SEQ ID NO: 31; a CDR2 region encoded by a nucleic acid sequence set forth in SEQ ID NO: 32; and a CDR3 region encoded by a nucleic acid sequence set forth in SEQ ID NO: 33. The nucleic acid is preferably an isolated nucleic acid. Optionally, the nucleic acid is cDNA.

The disclosure further provides a nucleic acid encoding an anti-relaxin antibody or functional fragment thereof, comprising a V_{L} domain comprising a CDR1 region encoded by a nucleic acid sequence set forth in SEQ ID NO: 40; a CDR2 region encoded by a nucleic acid sequence set forth in SEQ ID NO: 41; and a CDR3 region encoded by a nucleic acid sequence set forth in SEQ ID NO: 42; and a V_{H} domain comprising a CDR1 region encoded by a nucleic acid sequence set forth in SEQ ID NO: 37; a CDR2 region encoded by a nucleic acid sequence set forth in SEQ ID NO: 38; and a CDR3 region encoded by a nucleic acid sequence set forth in SEQ ID NO: 39. The nucleic acid is preferably an isolated nucleic acid. Optionally, the nucleic acid is cDNA.

In embodiments, a nucleic acid encodes an anti-relaxin antibody or functional fragment thereof, comprising a V_{H} domain encoded by a nucleic acid sequence set forth in SEQ ID NO: 43, and a V_{L} domain encoded by a nucleic acid sequence set forth in SEQ ID NO: 44. The nucleic acid is preferably an isolated nucleic acid. Optionally, the nucleic acid is cDNA.

In other embodiments, a nucleic acid encoding an anti-relaxin antibody or functional fragment thereof, comprising a V_{H} domain encoded by a nucleic acid sequence set forth in SEQ ID NO: 45, and a V_{L} domain encoded by a nucleic acid sequence set forth in SEQ ID NO: 46. The nucleic acid is preferably an isolated nucleic acid. Optionally, the nucleic acid is cDNA.

In still other embodiments, a nucleic acid encoding an anti-relaxin antibody or functional fragment thereof, comprising a V_{H} domain encoded by a nucleic acid sequence set forth in SEQ ID NO: 47, and a V_{L} domain encoded by a nucleic acid sequence set forth in SEQ ID NO: 48. The nucleic acid is preferably an isolated nucleic acid. Optionally, the nucleic acid is cDNA.

Further disclosed is a nucleic acid encoding an anti-relaxin antibody or a functional fragment thereof, comprising a V_{L} domain comprising a CDR1 region encoded by a nucleic acid sequence selected from the group consisting of sequences set forth in SEQ ID NOs: 28, 34 or 40; a CDR2 region encoded by a nucleic acid sequence selected from the group consisting of sequences set forth in SEQ ID NOs: 29, 35 or 41; and a CDR3 region encoded by a nucleic acid sequence selected from the group consisting of sequences set forth in SEQ ID NOs: 30, 36 or 42, and a V_{H} domain comprising a CDR1 region encoded by a nucleic acid sequence selected from the group consisting of sequences set forth in SEQ ID NOs: 25, 31 or 37; a CDR2 region encoded by a nucleic acid sequence selected from the group consisting of sequences set forth in SEQ ID NOs: 26, 32 or 38; and a CDR3 region encoded by a nucleic acid sequence selected from the group consisting of sequences set forth in SEQ ID NOs: 27, 33 or 39. The nucleic acid is preferably an isolated nucleic acid. Optionally, the nucleic acid is cDNA.

The disclosure moreover provides a nucleic acid encoding an anti-relaxin antibody or functional fragment thereof, comprising a V_{H} domain encoded by a nucleic acid sequence selected from the group consisting of sequences set forth in SEQ ID NOs: 43, 45 or 47, and a V_{L} domain encoded by a nucleic acid sequence selected from the group consisting of sequences set forth in SEQ ID NOs: 44, 46 or 48. The nucleic acid is preferably an isolated nucleic acid. Optionally, the nucleic acid is cDNA.

All nucleic acid and amino acid sequences of the present disclosure can comprise in addition to the sequences disclosed herein also their conservative sequence variants. The term “conservative sequence variant” as used herein, is intended to include nucleotide and amino acid sequence modifications, which do not significantly alter the binding properties of the anti-relaxin antibodies according to the present embodiments. Conservative nucleotide sequence variants include variants arising from the degeneration of the genetic code and from silent mutations. Nucleotide substitutions, deletions and additions are also included. Conservative amino acid sequence variants include variants arising from amino acid substitutions with similar amino acids well known in the art. Amino acid deletions and additions are also included.

When desired, DNA encoding the light and/or heavy chain CDRs or variable regions of the antibodies or functional fragment(s) thereof according to the present disclosure can be isolated and fused to the DNA encoding any desired constant region or modified constant region in order to produce a DNA construct which can be inserted into an expression vector or plasmid and transfected into a suitable expression host to produce a recombinant antibody. Thus, antibodies and functional fragment(s) thereof may also be produced in a host cell transfectoma using, for example, a combination of recombinant DNA techniques and gene transfection methods as is well known in the art.

For example, to express the antibodies, or functional fragments thereof, DNAs encoding partial or full-length light and heavy chains, can be obtained by standard molecular biology techniques (e.g., PCR amplification or cDNA cloning using a hybridoma that expresses the antibody of interest) and the DNAs can be inserted into expression vectors or plasmids such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector or plasmid and expression control sequences are chosen to be compatible with the expression host cell used. The antibody light chain gene and the antibody heavy chain gene can be inserted into separate vector or plasmid or, more typically, both genes are inserted into the same expression vector or plasmid. The antibody genes are inserted into the expression vector or plasmid by standard methods (e.g., ligation of complementary restriction sites on the antibody gene fragment and vector, or blunt end ligation if no restriction sites are present). The light and heavy chain variable regions of the antibodies described herein can be used to create full-length antibody genes of any antibody isotype by inserting them into expression vectors or plasmids already encoding heavy chain constant and light chain constant regions of the desired isotype such that the V_{H} segment is operatively linked to the heavy chain constant (C_{H}) segment(s) within the vector and the V_{L} segment is operatively linked to the light chain constant (C_{L}) segment within the vector. Additionally or alternatively, the recombinant expression vector or plasmid can encode a signal peptide that facilitates secretion of the antibody chain from a host cell. The antibody chain gene can be cloned into the vector or plasmid such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors or plasmids of some embodiments of the invention carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes, as well known in the art. It will be appreciated by those skilled in the art that the design of the expression vector or plasmid, including the selection of regulatory sequences, may depend on such factors as the choice of the host cell to be transformed, the level of expression of protein desired, etc.

In addition to the antibody chain genes and regulatory sequences, the recombinant expression vectors or plasmids of some embodiments may carry additional sequences, such as sequences that regulate replication of the vector in host cells (e.g., origins of replication) and selectable marker genes. As well known in the art, the selectable marker gene facilitates selection of host cells into which the vector or plasmid has been introduced.

For expression of the light and heavy chains, the expression vector(s) or plasmid(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies or functional fragment(s) thereof of the embodiments in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody.

Suitable mammalian host cells for expressing the recombinant antibodies or functional fragment(s) thereof of the invention include Chinese Hamster Ovary (CHO cells) (including dhfr-CHO cells know in the art), NSO myeloma cells, COS cells and SP2 cells. When recombinant expression vectors encoding antibody genes are introduced into mammalian host cells, the antibodies may be produced by culturing the host cells for a period of time sufficient to allow for expression of the antibody in the host cells or, more preferably, secretion of the antibody into the culture medium in which the host cells are grown. Antibodies may be recovered from the culture medium using standard protein purification methods.

Further provided are expression vectors comprising nucleotide sequences described herein. Suitable expression vectors include vectors containing elements important for the expression and secretion of proteins in mammalian host cells. The vector may comprise DNA encoding human heavy chain constant regions, or light chain constant regions, or both. The same vector may be used for the expression of both heavy and light chains or, alternatively, different vectors containing either heavy or light chain constant regions may be used.

The present disclosure still further provides host cells transfected with expression vectors or plasmids according to the present embodiments. Any suitable host cell/vector or plasmid system may be used for expression of the DNA sequences coding for the antibody heavy and light chains. Bacterial e.g. Escherichia coli, and other microbial systems may be used, in particular for expression of antibody fragments such as Fab and F(ab′)2 fragments, and especially Fv fragments and single chain antibody fragments e.g. single chain Fv's. Eukaryotic e.g. plant, yeast or mammalian host cell expression systems or transgenic plants and animals may be used for production of larger antibody products, including complete antibody molecules, and/or if glycosylated products are required. Suitable mammalian host cells include CHO (Chinese hamster ovary) cells and myeloma or hybridoma cell lines as set forth above. Preferred host cells are CHO cells.

The disclosure also provides hybridoma cell lines producing the anti-relaxin antibodies 2A4, 2H7 and 9B8. As described in the Deposit Statement herein, cultures of the hybridoma cell lines have been deposited with the China Center for Type Culture Collection (CCTCC) under Accession No:s CCTCC C2021210, CCTCC C2021209 and CCTCC C2021211 for the 2A4, 2H7 and 9B8 hybridoma cell lines, respectively.

The hybridoma cell line may be any mutant or variant of the 2A4, 2H7 or 9B8 hybridoma cell line that produces an anti-relaxin antibody retaining same or similar relaxin binding properties as 2A4, 2H7 or 9B8. For example, the cell line may produce an anti-relaxin antibody comprising a V_{L} domain comprising a CDR1 region having an amino acid sequence selected from the group consisting of sequences set forth in SEQ ID NOs: 4, 10 or 16; a CDR2 region having an amino acid sequence selected from the group consisting of sequences set forth in SEQ ID NOs: 5, 11 or 17; and a CDR3 region having an amino acid sequence selected from the group consisting of sequences set forth in SEQ ID NOs: 6, 12 or 18, and a V_{H} domain comprising a CDR1 region having an amino acid sequence selected from the group consisting of sequences set forth in SEQ ID NOs: 1, 7 or 13; a CDR2 region having an amino acid sequence selected from the group consisting of sequences set forth in SEQ ID NOs: 2, 8 or 14; and a CDR3 region having an amino acid sequence selected from the group consisting of sequences set forth in SEQ ID NOs: 3, 9 or 15. Alternatively, the cell line may produce any of the anti-relaxin antibodies or functional fragments thereof described herein.

Furthermore, the present disclosure provides a method for preparing the antibody or functional fragment of the present disclosure, comprising culturing the cell comprising a nucleic acid encoding the antibody or functional fragment described herein or a vector or plasmid comprising said nucleic acid in a medium under conditions that allow expression of the nucleic acid encoding the antibody or functional fragment, and optionally recovering the antibody or functional fragment from the cells or from the medium. General methods by which the vectors may be constructed, transfection methods and culture methods are well known in the art.

The following examples are given to further clarify the embodiments of the invention in more detail but are not intended to restrict the scope of the present invention. Further applications and uses are readily apprehended by a person skilled in the art.

### EXAMPLES

Monoclonal IgG antibodies were prepared by immunizing mice with preprorelaxin (Bioraytec Ltd., Zhuhai, China) using standard laboratory methods known in the art. Anti-preprorelaxin antibody expressing hybridomas were derived by fusing myeloma cells with splenocytes from the immunized mice. The antibodies produced by hybridoma clonal cell lines were initially screened for activity against relaxin-positive pregnant dog serum. Relaxin positivity of the serum was confirmed using the FASTest RELAXIN in vitro test (Diagnostik Megacor, Hörbranz, Austria). The serum contains mature relaxin as preprorelaxin is not present in serum, and so the selected antibodies are reactive against relaxin and not only preprorelaxin.

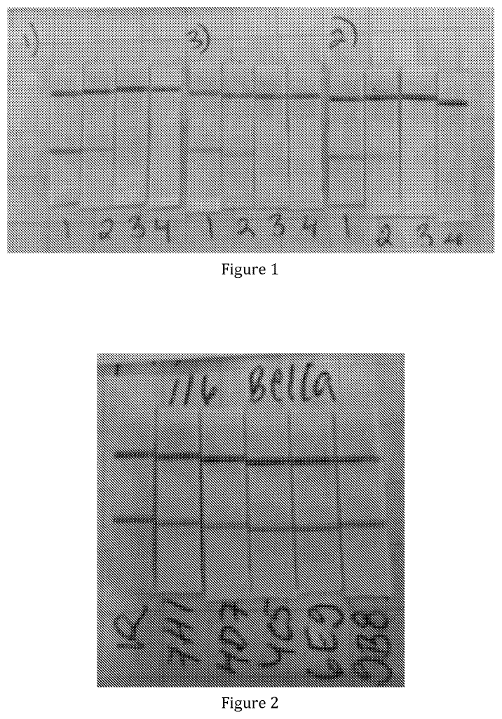

The anti-relaxin antibodies showing activity were further tested to select capture and detection antibody pairs for use in the relaxin test for pregnancy. Anti-relaxin antibodies 2H7, 10B11, 6C12, 2F10 and 2E12 resulted from the first immunization. In preliminary tests performed with all 20 possible capture-detection antibody combinations, the best results (not shown) were given by the combinations having 2H7 as the capture or detection antibody. Further tests were performed with five different combinations (2H7-10B11, 2H7-6C12, 2H7-2F10, 2H7-2E12 and 2F10-2H7). Of these, the three most promising combinations were 2H7-2E12, 2H7-10B11 and 2F10-2H7. Results of tests performed with these combinations are shown in FIG. 1.

In FIG. 1, 1) is the 2H7-2E12 pair with 2H7 as capture and 2E12 as detection antibody, 3) is the 2H7-10B11 pair with 2H7 as capture and 10B11 as detection antibody and 2) is the 2F10-2H7 pair with 2F10 as capture and 2H7 as detection antibody. The upper line in each test strip is a control line showing that the test has been performed successfully. The control line comprises a polyclonal anti-mouse antibody which binds to the murine detection antibody which is labelled by coating on a gold nanoparticle having a 35-40 nm diameter. The lower line is a test line showing the test result which is positive for relaxin if a line is visible and negative for relaxin if no line is visible. The gold nanoparticle-labeled detection antibody is present at that end of the test strip which is closer to the test line and is placed in a conjugate pad which is adjoined to the nitrocellulose membrane test strip with a 1-2 mm overlap. The sample or control is applied to a sample pad adjoining the conjugate pad containing the detection antibody, and the sample with the detection antibody migrates by lateral flow towards the test line. If relaxin is present in the sample, it forms a complex with the detection antibody. The capture antibody is present in the test line and captures by binding the relaxin-detection antibody complex to form a visible test line. The detection antibody that is not in complex with relaxin continues to migrate towards the control line. If no relaxin is present in the sample, all of the detection antibody migrates past the test line towards the control line, where it is bound by the anti-mouse antibody to form a visible control line. Thus, two lines indicates a positive test result for relaxin, and one line (the control line) indicates a negative result. The samples are from three different dogs (1=Bella, 2=Ebba, 3=Noita) and a control sample (4) which is a PBS buffer solution (Phosphate Buffered Saline (PBS); obtained as 20× concentrate pH 7.5 from VWR and diluted to 1×PBS). Samples 1 and 2 are from pregnant dogs and sample 3 is a negative control from a non-pregnant dog.

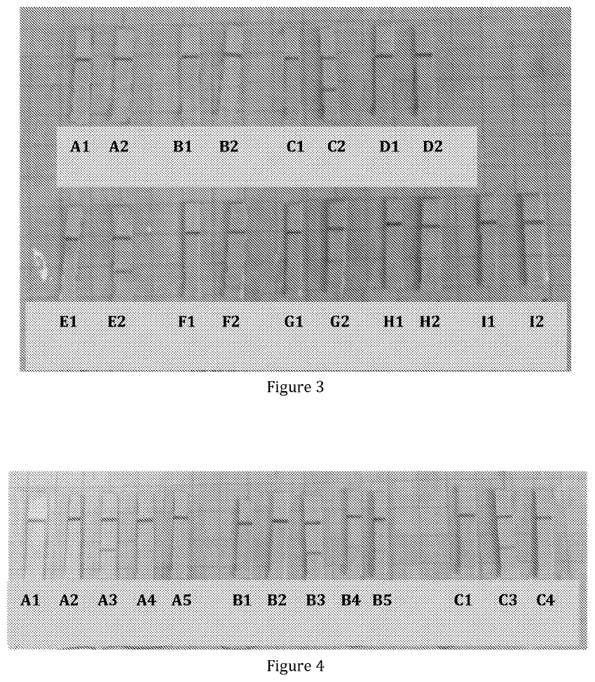

A second immunization of mice with preprorelaxin resulted in anti-relaxin antibodies 7H1, 4D7, 4C5, 6F9, 9B8, 7G7, 5H11, 3A7, 2A4. Again, these antibodies were selected based on their reactivity with relaxin-positive female dog serum. In preliminary tests (results not shown), in which the antibody 2F10 from the first immunization was also included, the antibodies were categorized into three groups: those that function as a pair with 2H7, those that function as a pair with 2E12 and those that do not have any specific recommended pair. The preliminary tests were performed to limit the number of antibody pairs to be tested later when screening for the most promising antibody pairs. In the screening tests, the following twelve antibody combinations were shown to react with pregnant dog serum: 2H7-7H1, 2H7-4D7, 2H7-4C5, 2H7-6E9, 2H7-9B8, 2H7-7G7, 2H7-2A4, 2H7-2F10, 2E12-3A7, 2E12-7G7, 2E12-5H11, 2E12-2F10. The results of screening are shown in FIGS. 2 and 3.

In FIG. 2, the capture antibody 2H7 is paired with detection antibody 7H1, 4D7, 4C5, 6E9 or 9B8. The first test strip from the left is a reference antibody pair 2H7-10B11/2E12. The reference antibody pair had two detection antibodies 10B11 and 2E12, both of which had been shown to produce a functioning test as seen from the results of FIG. 1. The sample in all tests of FIG. 2 is sample 1 (Bella) which had been diluted ⅙ in 1×PBS buffer solution using 1 volume sample and 5 volumes 1×PBS buffer. All five pairs were found to give a visible test line.

In FIG. 3, results of capture antibody—detection antibody pairs 2H7-7G7 (A), 2H7-5H11 (B), 2H7-2A4 (C), 2H7-2F10 (D) 2E12-3A7 (E), 2E12-10B11/2E12 (F), 2E12-7G7 (G), 2E12-5H11 (H) and 2E12-2F10 (I). Control (1) is 1×PBS buffer and positive sample (2) in all tests is pregnant dog serum (Bella) which has been diluted ⅙ in PBS buffer solution using 1 volume sample and 5 volumes 1×PBS buffer. Antibody pairs 2H7-5H11 and 2E12-10B11/2E12 did not show a visible test line and were considered non-applicable in the assay.

All 12 antibody pairs (21H 7-7H1, 2H7-4D7, 2H7-4C5, 2H7-6E9, 2H7-9B8, 2H7-7G7, 2H7-2A4, 2H7-2F10, 2E12-3A7, 2E11-7G7, 2E12-2F10) selected in screening tests, had either 2H7 or 2E12 as capture antibody. 2H7 gave a larger number of antibody pairs with a strongly visible test line than 2E12 (results not shown), and so 2H7 is preferably used as a capture antibody.

Sensitization of the relaxin pregnancy test was studied by using two antibodies as a detection antibody pair with capture antibody 2H7. The results are shown in FIG. 4. The antibody combinations used were 2H7-2A4/7H1 (A), 2H7-2A4/9B8 (B), 2H7-7H1/9B8 (C). Control (1) was 1×PBS buffer, negative control (2) was serum from a non-pregnant female dog (Noita) and positive samples were pregnant dog serums Bella (3), Honey (4) and Macy (5).

The combination 2H7-7H1/9B8 in the assay caused background color formation, which was caused by aggregation upon gold-conjugating the antibodies. Therefore, this antibody combination was eliminated from further testing. Of the two remaining antibody combinations 2H7-2A4/9B8 was selected for use in the tests as it did not show aggregation upon gold-conjugation and showed high sensitivity by giving positive test results i.e. a visible test line even in samples collected at 20 or 21 days from breeding.

The 2H7-2A4/9B8 test was used for analysing a panel of dog serum samples representing days 0-53 post-breeding (post-ovulation). Some of the samples were collected from the same dog after two breedings performed on different dates, with the first breeding turning out to be unsuccessful (Lulu I and Lulu II; Armi I and Armi II; Noita I and Noita II), so the samples were from 11 different female dog individuals. The tests were performed on test strips as described above. The sera were diluted 1:4 (one volume serum, four volumes 1×PBS buffer) or 1:6 (one volume serum, six volumes 1×PBS buffer) prior to the assay. All 1:4 dilutions of samples from 21 or more days post-breeding (post-ovulation) were tested as duplicates, so the result in Table 1 is the average value of these duplicate assays. In the assay using 1:6 dilutions the intensity of the control line was decreased from that used with 1:4 dilutions by decreasing the amount of anti-mouse antibody to reduce background level. The test results were recorded from the test strips at 10 min from applying the sample using the Findout application on a mobile phone. The Findout application determines the ratio of intensity between the test and control line, where a result 0 indicates that there is detectable color only in the control line and no test line is detectable; result 5 indicates the test line and control line are equal in intensity; and result 10 indicates there is detectable color only in the test line and no control line is detectable. Generally, at a value of about 0.2-0.3 the test line is barely visible with the naked eye, and a result that can be considered positive requires a value of about 0.8-1.0. The results are shown in Table 1. The results show that relaxin level as determined with the test correlates with the duration of pregnancy, with relaxin level increasing as the gestation progresses. An increase in relaxin level i.e. a positive pregnancy test result is seen as early as at 20 days from breeding.

TABLE 1
2H7-2A4/9B8 test results are indicated as ratio of test line intensity
to control line intensity (0 = no test line detectable; 5 =
equal intensity in test line and color line; 10 = no control line detectable).

| | Days from breeding | | | | | | | |
|---|---|---|---|---|---|---|---|---|
| Dog | 0 | 14 | 20 | 21 | 26 | 28 | 48 | 53 |
| Noita 1 1:4 | 0.25 | | | | | | | |
| Noita I 1:6 | 0.18 | | | | | | | |
| Noita II 1:6 | 0.06 | | | | | | | |
| Lulu I 1:4 | 0.32 | | | | | | | |
| Lulu I 1:6 | 0.04 | | | | | | | |
| Lulu II 1:4 | 0.18 | | | | | | | |
| Armi I 1:4 | 0.28 | | | | | 3.26 | | |
| Armi I 1:6 | 0.32 | | | | | 2.00 | | |
| Armi II 1:6 | 0.05 | | | | | | | |
| Ruu 1:4 | 0.36 | | | | | | | |
| Ruu 1:6 | 0.52 | | | | | | | |
| Piper 1:4 | | 0.31 | | | | | | |
| Piper 1:6 | 0.72 | 0.08 | | | | | | |
| Ebba 1:4 | | | 1.16 | | | | 4.87 | |
| Ebba 1:6 | | | 0.85 | | | | 3.81 | |
| Macy 1:4 | | | | 1.31 | | | | |
| Macy 1:6 | 0.43 | | | 0.45 | | | | |
| Honey 1:4 | | | | | 3.24 | | | |
| Honey 1:6 | 0.36 | | | | 1.77 | | | |
| Bella 1:4 | | | | | | | | 5.09 |
| Bella 1:6 | | | | | | | | 4.74 |
| Alma 1:6 | 0.15 | | | | | | | |
| Hemu 1:6 | 0.77 | | | | | | | |

The 2H7-2A4/9B8 test was also performed on a panel of dog serum samples which have a known progesterone content. These samples originated from a veterinary clinic (Evidensia, Mantsala, Finland), and pregnancy status or gestation day of the dogs was not known. Progesterone assays can be useful in monitoring the pregnancy since progesterone is a hormone required in bitches to maintain pregnancy. Progesterone values above 5 ng/ml during pregnancy are assuring to some extent; those below 4-5 ng/ml any time between days 30-55 after the preovulatory luteinizing hormone (LH) surge may indicate luteal insufficiency. Progesterone concentrations peak at 15-90 ng/ml sometime between day 15 and 25 after the LH surge in both pregnancy and non-pregnant bitches. The test results were recorded from the test strips at 10 min from applying the sample using the Findout application on a mobile phone. The results are shown in Table 2. The results indicate there is no cross-reactivity with progesterone or other interference by progesterone in the 2H7-2A4/9B8 assay.

TABLE 2
Performance of the 2H7-2A4/9B8 in samples with known progesterone
content. 2H7-2A4/9B8 test results are indicated as ratio
of test line intensity to control line intensity (0 =
no test line detectable; 5 = equal intensity in test
line and color line; 10 = no control line detectable).

| Sample no. | Progesterone content (ng/ml) | 2H7-2A4/9B8 test result |
|---|---|---|
| 3 | 5.21 | 0.16 |
| 4 | 4.7 | 0.31 |
| 5 | 4.33 | 2.47 |
| 6 | 3.81 | 2.46 |
| 7 | 18.2 | 0.29 |
| 8 | 1.17 | 1.27 |
| 9 | 2.43 | 0.33 |
| 10 | 1.60 | 0.28 |
| 11 | 0.78 | 0.30 |
| 12 | 5.76 | 0.19 |
| 13 | 1.97 | 1.23 |

The 2H7-2A4/9B8 test was also shown to function with whole blood samples. Red blood cells were essentially filtered out from the whole blood as the sample passed through the test pads, so that as the sample migrated to the nitrocellulose strip it essentially comprised of plasma. Dog plasma behaved similarly as sample as dog serum.

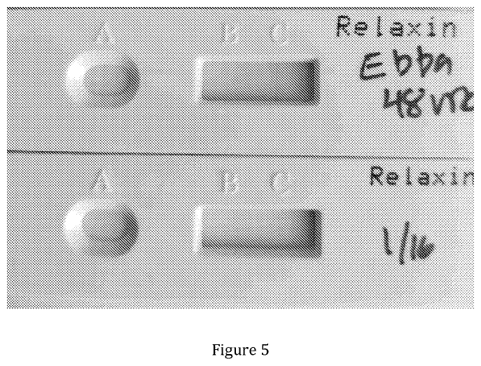

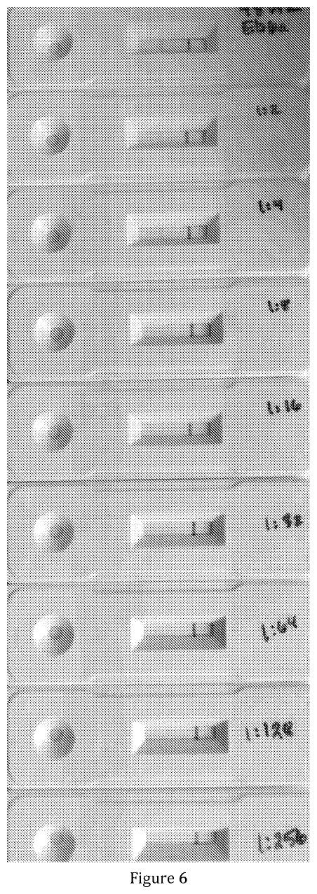

Performance of the 2H7-2A4/9B8 test was compared with the commercial FASTest RELAXIN test (Diagnostik Megacor, Hörbranz, Austria). With the 2H7-2A4/9B8 test, assay of a pregnant dog serum sample (Ebba, 48 d post-breeding) was performed with dilutions made into 1×PBS buffer. One part of sample was diluted with 1, 2, 4, 8, 16, 32, 64, 128, or 256 parts of buffer. The 2H7-2A4/9B8 test was performed with all sample dilutions. FASTest RELAXIN test was performed according to manufacturer's instructions using 1:1 and 1:16 sample dilutions. The results were recorded 10 min after applying the sample and are illustrated in FIGS. 5 and 6. FIG. 5 shows test results of FASTest RELAXIN test with 1:1 dilution above and 1:16 dilution below. No test line was visible at position B of the test strip with either of the dilutions. A control line was visible at position C with both dilutions, although the control line was less pronounced with the 1:16 dilution. FIG. 6 shows test results of 2H7-2A4/9B8 test with dilutions in the order of 1:1, 1:2, 1:4 1:8, 1:16, 1:32, 1:64, 1:128 and 1:256 starting from top. A control line was clearly visible with all dilutions. The test line was clearly discernible with 1:32 dilution, and a faint test line could be seen with the naked eye also with the 1:64, 1:128 and 1:256 dilutions. The results show that the 2H7-2A4/9B8 test is more sensitive than the commercial FASTest RELAXIN test.

It will be obvious to a person skilled in the art that, as the technology advances, the inventive concept can be implemented in various ways. The invention and its embodiments are not limited to the examples described above but may vary within the scope of the claims.

### Deposit Statement

Cultures of the following biological material(s) have been deposited with the following international depository:

### China Center for Type Culture Collection (CCTCC)

### Wuhan University

### Wuhan 430072

### P.R. China

under conditions that satisfy the requirements of the Budapest Treaty on the International Recognition of the Deposit of Microorganisms for the Purposes of Patent Procedure.

### International Depository Accession

| Hybridoma Deposited | Accession No. | Date of Deposit |
|---|---|---|
| 2A4 | CCTCC C2021210 | Aug. 31, 2021 |
| 2H7 | CCTCC C2021209 | Aug. 31, 2021 |
| 9B8 | CCTCC C2021211 | Aug. 31, 2021 |

1. An assay method for relaxin, wherein the method comprises
(a) binding relaxin with a capture antibody comprising
a VL domain comprising a CDR1 region having an amino acid sequence set forth in SEQ ID NO: 10, a CDR2 region having an amino acid sequence set forth in SEQ ID NO: 11, and a CDR3 region having an amino acid sequence set forth in SEQ ID NO: 12, and a VH domain comprising a CDR1 region having an amino acid sequence set forth in SEQ ID NO: 7, a CDR2 region having an amino acid sequence set forth in SEQ ID NO: 8, and a CDR3 region having an amino acid sequence set forth in SEQ ID NO: 9;
or a functional fragment thereof which has independently at least 80% sequence identity, or at least 85%, 90%, 95%, 96%, 97%, 98% or 99% sequence identity to any one of SEQ ID NO: 7-12, wherein the functional fragment is capable of binding relaxin, in particular canine or feline relaxin, preferably canine relaxin;
and
(b) binding relaxin with a first detection antibody comprising
a VL domain comprising a CDR1 region having an amino acid sequence set forth in SEQ ID NO: 4, a CDR2 region having an amino acid sequence set forth in SEQ ID NO: 5, and a CDR3 region having an amino acid sequence set forth in SEQ ID NO: 6, and a VH domain comprising a CDR1 region having an amino acid sequence set forth in SEQ ID NO: 1, a CDR2 region having an amino acid sequence set forth in SEQ ID NO: 2, and a CDR3 region having an amino acid sequence set forth in SEQ ID NO: 3;
or a functional fragment thereof which has independently at least 80% sequence identity, or at least 85%, 90%, 95%, 96%, 97%, 98% or 99% sequence identity to any one of SEQ ID NO: 1-6, wherein the functional fragment is capable of binding relaxin, in particular canine or feline relaxin, preferably canine relaxin;
and
(c) binding relaxin with a second detection antibody comprising
a VL domain comprising a CDR1 region having an amino acid sequence set forth in SEQ ID NO: 16, a CDR2 region having an amino acid sequence set forth in SEQ ID NO: 17, and a CDR3 region having an amino acid sequence set forth in SEQ ID NO: 18, and a VH domain comprising a CDR1 region having an amino acid sequence set forth in SEQ ID NO: 13, a CDR2 region having an amino acid sequence set forth in SEQ ID NO: 14, and a CDR3 region having an amino acid sequence set forth in SEQ ID NO: 15;
or a functional fragment thereof which has independently at least 80% sequence identity, or at least 85%, 90%, 95%, 96%, 97%, 98% or 99% sequence identity to any one of SEQ ID NO: 13-18, wherein the functional fragment is capable of binding relaxin, in particular canine or feline relaxin, preferably canine relaxin.

2. An assay method for relaxin, wherein the method comprises
(a) binding relaxin with a capture antibody comprising
a VH domain having an amino acid sequence set forth in SEQ ID NO: 21, and a VL domain having an amino acid sequence set forth in SEQ ID NO: 22;
or a functional fragment thereof which has independently at least 80% sequence identity, or at least 85%, 90%, 95%, 96%, 97%, 98% or 99% sequence identity to any one of SEQ ID NO: 21 or 22, wherein the functional fragment is capable of binding relaxin, in particular canine or feline relaxin, preferably canine relaxin;
and
(b) binding relaxin with a first detection antibody comprising
a VH domain having an amino acid sequence set forth in SEQ ID NO: 19, and a VL domain having an amino acid sequence set forth in SEQ ID NO: 20;
or a functional fragment thereof which has independently at least 80% sequence identity, or at least 85%, 90%, 95%, 96%, 97%, 98% or 99% sequence identity to any one of SEQ ID NO: 19 or 20, wherein the functional fragment is capable of binding relaxin, in particular canine or feline relaxin, preferably canine relaxin;
and
(c) binding relaxin with a second detection antibody comprising
a VH domain having an amino acid sequence set forth in SEQ ID NO: 23, and a VL domain having an amino acid sequence set forth in SEQ ID NO: 24;
or a functional fragment thereof which has independently at least 80% sequence identity, or at least 85%, 90%, 95%, 96%, 97%, 98% or 99% sequence identity to any one of SEQ ID NO: 23 or 24, wherein the functional fragment is capable of binding relaxin, in particular canine or feline relaxin, preferably canine relaxin.

3. The method according to claim 1, wherein said antibody or functional fragment comprises
(i) a VL domain comprising a CDR1 region having an amino acid sequence set forth in SEQ ID NO: 4; a CDR2 region having an amino acid sequence set forth in SEQ ID NO: 5; and a CDR3 region having an amino acid sequence set forth in SEQ ID NO: 6; and a VH domain comprising a CDR1 region having an amino acid sequence set forth in SEQ ID NO: 1; a CDR2 region having an amino acid sequence set forth in SEQ ID NO: 2; and a CDR3 region having an amino acid sequence set forth in SEQ ID NO: 3; or
(ii) a VL domain comprising a CDR1 region having an amino acid sequence set forth in SEQ ID NO: 10; a CDR2 region having an amino acid sequence set forth in SEQ ID NO: 11; and a CDR3 region having an amino acid sequence set forth in SEQ ID NO: 12; and a VH domain comprising a CDR1 region having an amino acid sequence set forth in SEQ ID NO: 7; a CDR2 region having an amino acid sequence set forth in SEQ ID NO: 8; and a CDR3 region having an amino acid sequence set forth in SEQ ID NO: 9; or
(iii) a VL domain comprising a CDR1 region having an amino acid sequence set forth in SEQ ID NO: 16; a CDR2 region having an amino acid sequence set forth in SEQ ID NO: 17; and a CDR3 region having an amino acid sequence set forth in SEQ ID NO: 18; and a VH domain comprising a CDR1 region having an amino acid sequence set forth in SEQ ID NO: 13; a CDR2 region having an amino acid sequence set forth in SEQ ID NO: 14; and a CDR3 region having an amino acid sequence set forth in SEQ ID NO: 15;
and wherein the functional fragment thereof is a functional fragment of any one of said anti-relaxin antibody (i), (ii) or (iii), which has 100% sequence identity to the VL and VH CDR1, CDR2 and CDR3 regions comprised therein, and which has independently at least 80% sequence identity, or at least 85%, 90%, 95%, 96%, 97%, 98% or 99% to any one of SEQ ID NO: 19, 20, 21, 22, 23 or 24 outside of said V_{L} and V_{H}CDR1, CDR2 and CDR3 regions.

4. The method of claim 1, wherein the functional fragment is a conservative sequence variant of any of the anti-relaxin antibodies.

5. The method of claim 1, wherein the relaxin is canine or feline relaxin, preferably canine relaxin.

6. A kit for pregnancy testing in an animal, wherein the kit comprises the capture antibody and the detection antibodies or functional fragments thereof as defined in claim 1, optionally wherein the animal is a dog or a cat.

7. An anti-relaxin antibody or functional fragment thereof, wherein said antibody or functional fragment comprises
(i) a VL domain comprising a CDR1 region having an amino acid sequence set forth in SEQ ID NO: 10, a CDR2 region having an amino acid sequence set forth in SEQ ID NO: 11, and a CDR3 region having an amino acid sequence set forth in SEQ ID NO: 12, and a VH domain comprising a CDR1 region having an amino acid sequence set forth in SEQ ID NO: 7, a CDR2 region having an amino acid sequence set forth in SEQ ID NO: 8, and a CDR3 region having an amino acid sequence set forth in SEQ ID NO: 9;
or a functional fragment thereof which has independently at least 80% sequence identity, or at least 85%, 90%, 95%, 96%, 97%, 98% or 99% sequence identity to any one of SEQ ID NO: 7-12, wherein the functional fragment is capable of binding relaxin, in particular canine or feline relaxin, preferably canine relaxin;
or
(ii) a VL domain comprising a CDR1 region having an amino acid sequence set forth in SEQ ID NO: 4, a CDR2 region having an amino acid sequence set forth in SEQ ID NO: 5, and a CDR3 region having an amino acid sequence set forth in SEQ ID NO: 6, and a VH domain comprising a CDR1 region having an amino acid sequence set forth in SEQ ID NO: 1, a CDR2 region having an amino acid sequence set forth in SEQ ID NO: 2, and a CDR3 region having an amino acid sequence set forth in SEQ ID NO: 3;
or a functional fragment thereof which has independently at least 80% sequence identity, or at least 85%, 90%, 95%, 96%, 97%, 98% or 99% sequence identity to any one of SEQ ID NO: 1-6, wherein the functional fragment is capable of binding relaxin, in particular canine or feline relaxin, preferably canine relaxin;
or
(iii) a VL domain comprising a CDR1 region having an amino acid sequence set forth in SEQ ID NO: 16, a CDR2 region having an amino acid sequence set forth in SEQ ID NO: 17, and a CDR3 region having an amino acid sequence set forth in SEQ ID NO: 18, and a VH domain comprising a CDR1 region having an amino acid sequence set forth in SEQ ID NO: 13, a CDR2 region having an amino acid sequence set forth in SEQ ID NO: 14, and a CDR3 region having an amino acid sequence set forth in SEQ ID NO: 15;
or a functional fragment thereof which has independently at least 80% sequence identity, or at least 85%, 90%, 95%, 96%, 97%, 98% or 99% sequence identity to any one of SEQ ID NO: 13-18, wherein the functional fragment is capable of binding relaxin, in particular canine or feline relaxin, preferably canine relaxin.

8. An anti-relaxin antibody or functional fragment thereof, wherein said antibody or functional fragment comprises
(i) a VH domain having an amino acid sequence set forth in SEQ ID NO: 19, and a VL domain having an amino acid sequence set forth in SEQ ID NO: 20, or
(ii) a VH domain having an amino acid sequence set forth in SEQ ID NO: 21, and a VL domain having an amino acid sequence set forth in SEQ ID NO: 22, or
(iii) a VH domain having an amino acid sequence set forth in SEQ ID NO: 23, and a VL domain having an amino acid sequence set forth in SEQ ID NO: 24;
and wherein the functional fragment thereof has independently at least 80% sequence identity, or at least 85%, 90%, 95%, 96%, 97%, 98% or 99% sequence identity to any one of SEQ ID NO: 19, 20, 21, 22, 23, or 24, and wherein the functional fragment thereof is capable of binding relaxin, in particular canine or feline relaxin, preferably canine relaxin.

9. The anti-relaxin antibody or functional fragment thereof of claim 7, wherein said antibody or functional fragment comprises
(i) a VL domain comprising a CDR1 region having an amino acid sequence set forth in SEQ ID NO: 4; a CDR2 region having an amino acid sequence set forth in SEQ ID NO: 5; and a CDR3 region having an amino acid sequence set forth in SEQ ID NO: 6; and a VH domain comprising a CDR1 region having an amino acid sequence set forth in SEQ ID NO: 1; a CDR2 region having an amino acid sequence set forth in SEQ ID NO: 2; and a CDR3 region having an amino acid sequence set forth in SEQ ID NO: 3; or
(ii) a VL domain comprising a CDR1 region having an amino acid sequence set forth in SEQ ID NO: 10; a CDR2 region having an amino acid sequence set forth in SEQ ID NO: 11; and a CDR3 region having an amino acid sequence set forth in SEQ ID NO: 12; and a VH domain comprising a CDR1 region having an amino acid sequence set forth in SEQ ID NO: 7; a CDR2 region having an amino acid sequence set forth in SEQ ID NO: 8; and a CDR3 region having an amino acid sequence set forth in SEQ ID NO: 9; or
(iii) a VL domain comprising a CDR1 region having an amino acid sequence set forth in SEQ ID NO: 16; a CDR2 region having an amino acid sequence set forth in SEQ ID NO: 17; and a CDR3 region having an amino acid sequence set forth in SEQ ID NO: 18; and a VH domain comprising a CDR1 region having an amino acid sequence set forth in SEQ ID NO: 13; a CDR2 region having an amino acid sequence set forth in SEQ ID NO: 14; and a CDR3 region having an amino acid sequence set forth in SEQ ID NO: 15;
and wherein the functional fragment thereof is a functional fragment of any one of said anti-relaxin antibody (i), (ii) or (iii), which has independently at least 80% sequence identity, or at least 85%, 90%, 95%, 96%, 97%, 98% or 99% to any one of SEQ ID NO: 19, 20, 21, 22, 23 or 24 outside of said V_{L} and V_{H}CDR1, CDR2 and CDR3 regions.

10. The anti-relaxin antibody or functional fragment thereof of claim 7, wherein the functional fragment thereof is a conservative sequence variant of any of the anti-relaxin antibodies.

11. A nucleic acid encoding the antibody or functional fragment according to claim 7, optionally wherein the nucleic acid is cDNA.

12. A vector or plasmid comprising the nucleic acid according to claim 11.

13. A cell comprising the nucleic acid according to claim 11 or the vector or plasmid.

14. A method of preparing the antibody or functional fragment according to claim 7, comprising culturing the cell in a medium under conditions that allow expression of the nucleic acid encoding the antibody or functional fragment, and optionally recovering the antibody or functional fragment from the cells or from the medium.

15. A cell line producing an anti-relaxin antibody, wherein the cell line is selected from
(i) CCTCC C2021209,
(ii) CCTCC C2021210, or
(iii) CCTCC C2021211.
